SEQUENCE LISTING (30 sequences)

SEQ ID NO: 1 - DNA, 1860 bp, Homo sapiens
tccgcctgcgctcctcagcctgacggtccgcctttcggggctcctcagccttgtcacccgctcttggttttccttttctcttcatctttggctcctttgaccactcgaagccgcgcagcgggttccagcggacctcacagcagccccagaagtggtgcgccaagcacagcctctgctcctcctcgagccggtcgggaactgctgcctgccgccatcatgtctactgcaaatcctgaaactccaaactcaaccatctccagagaggccagcacccagtcctcatcagctgcaaccagccaaggctatattttaccagaaggcaaaatcatgccaaacactgtttttgttggaggaattgatgttaggatggatgaaactgagattagaagcttctttgctagatatggttcagtgaaagaagtgaagataatcactgatcgaactggtgtgtccaaaggctatggatttgtttcattttttaatgacgtggatgtgcagaagatagtagaatcacagataaatttccatggtaaaaagctgaagctgggccctgcaatcaggaaacaaaatttatgtgcttatcatgtgcagccacgtcctttggtttttaatcatcctcctccaccacagtttcagaatgtctggactaatccaaacactgaaacttatatgcagcccacaaccacgatgaatcctataactcagtatgttcaggcatatcctacttacccaaattcaccagttcaggtcatcactggatatcagttgcctgtatataattatcagatgccaccacagtggcctgttggggagcaaaggagctatgttgtacctccggcttattcagctgttaactaccactgtaatgaagttgatccaggagctgaagttgtgccaaatgaatgttcagttcatgaagctactccaccctctggaaatggcccacaaaagaaatctgtggaccgaagcatacaaacggtggtatcttgtctgtttaatccagagaacagactgagaaactctgttgttactcaagatgactacttcaaggataaaagagtgcatcactttagaagaagtcgggcaatgcttaaatctgtttgatcctcctggcttatctagttacatgggaagttgctggttttgaatattaagctaaaaggtttccactattatagaaattctgaattttggtaaatcacactcaaactttgtgtataagttgtattattagactctctagttttatcttaaactgttcttcattagatgtttatttagaaactggttctgtgttgaaatatagttgaaagtaaaaaaataattgagactgaaagaaactaagatttatctgcaaggattttttaaaaattggcattttaagtgtttaaaagcaaatactgattttcaaaaaaatgtttttaaaaacctattttgaaaggtcagaattttgttggtctgaatacaaacatttcacttctccaacaagtacctgtgaacagtacagtatttacagtattgagctttgcatttatgatttctccagaaatttaccacaaaagcaaaatttttaaaactgcatttttaatcagtggaactcaatatatagttagctttattgaagtcttcttatctaaacccagcaaaacagattcaaagcgaacagtccaatcagtgggtcatatgtttattcaaaatattttatcttttagctagaatccacacatatatatcctatttgattarggtagtaattagataactaaaattctgggcctaattttttaaagaatccmagacaaactaaactttactaggtacataagcttctccatgaatcaccatcctcctttttggtaa

SEQ ID NO: 2 - protein, 295 aa, Homo sapiens
MSTANPETPNSTISREASTQSSSAATSQGYILPEGKIMPNTVFVGGIDVRMDETEIRSFFARYGSVKEVKIITDRTGVSKGYGFVSFFNDVDVQKIVESQINFHGKKLKLGPAIRKQNLCAYHVQPRPLVFNHPPPPQFQNVWTNPNTETYMQPTTTMNPITQYVQAYPTYPNSPVQVITGYQLPVYNYQMPPQWPVGEQRSYVVPPAYSAVNYHCNEVDPGAEVVPNECSVHEATPPSGNGPQKKSVDRSIQTVVSCLFNPENRLRNSVVTQDDYFKDKRVHHFRRSRAMLKSV

SEQ ID NO: 3 - DNA, 1207 bp, Homo sapiens
tctaaggaaattcaccgaaaacctccagtagccacctgctctttgccctgacagttctcaaggaagtggtcctcctgctgccccaagcctgtcagcctccatgaaaccaagccttccacactataatacagaaaagtaaagtctttgctttccgggctaccttgtagcaatttgaggctctgtcatcagtttctgctacgtttcaaagatccaggagaagcttagtgttgtgtcaagacgccgatggacccatcacagtttaatccaacctacaacccagggtctccacaaatgctcaccgaagaaaattcccgggacgattcaggggcctctcaaatctcctccgagacgttgataaagaaccttagtaacttgactatcaacgctagtagcgaatctgtttcccctctattggaagctttactccgtcgagagtctgtgggggcagcagtcctcagggaaatcgaagatgagtggctttacagcaggagaggagtaagaacactgctgtctgtgcagagagaaaagatggcaagattgagatacatgttactgggcggagttcgtacgcatgaaagaagaccaacaaacaaggagcctaagggagttaagaaggaatcaagaccattcaaatgtccctgcagtttctgcgtgtctaatggatgggatccttctgagaatgctagaatagagaatcaagacaccaagccacttcagccataaatcttattcttgcacctttttttcttgctagtaattttatatagcaggttgagaaagctactctatgctagtatagactatacaccaataattttgataatgagttctaggatgtatttttcttgtatctttttcttcctactatgatactagtaattcataagggatctgtgtaatctgaatgtatttcaataactttagctctactgtttgatttgacccaaagaagccaagacgatataagtattcccatgtgtcttagaagcccaaagtcagtgagatgaaacccaacatcaagaaattgaagcaaagttacttgtggataaagaaagcattaggtagtttggctatagcataattagattttctggctttcaaaaatttggattgcaatcacagcaaactttgttatttttacagttttcagtacaaaagtgtttatatagaaacaataaagttgacatttgagtacctttaaaaaaaa

SEQ ID NO: 4 - DNA, 1085 bp, Homo sapiens
cacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaaaaa

SEQ ID NO: 5 - DNA, 1599 bp, Homo sapiens
aacatttccaaatcttggcattcttatccacaaagtgaagataataattgtcaattcacaggtgattatgatttaaagagattacttttgaagagttcctaacacattcagtcaacatttaatgatgcttcaggcactgtgttcattgctagtgagcgtatgacacacacagccatacggtcacagagctttcaatgaaaagtaacataattgctcatttcaccaggcccccggcttggggcgccttccttccccatggcgggacacctggcttcggatttcgccttctcgccccctccaggcggtgggggtgatgggccatggggggcggagccgggctgggttgatcctctgacctggctaagcttccaaggccctcctggagggccaggaatcgggccgggggttgggccaggctctgaggtgtgggggattcccccttgccccccgccgtatgagttatgtggggggatggcgtactgtgggcctcaggttggagtggggctagtgccccaaggcggcttggagacctctcagcctgagagcgaagcaggagtcggggtggagagcaactccaatggggcctccccggaaccctgcaccgtcccccctggtgccgtgaagctggagaaggagaagctagagcaaaacccggagaagtcccaggacatcaaagctctgcagaaagaactcgagcaatttgccaagctcctgaagcagaagaggatcaccctgggatatacacaggccgatgtggggctcatcctgggggttctatttgggaaggtgttcagccaaaagaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcatgcaggcccgaaagagaaagcgaaccagtatcgagaaccgagtgagaggcaacctggagaatttgttcctgcagtgcccgaaacccacactgcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctccggccccagggccccattttggtaccccaggctatgggagccctcacttcactgcactgtactcctcagtccctttccctgagggggaagtctttcccccagtctccgtcatcactctgggctctcccatgcattcaaactgaggtgcctgcccttctaggaatggggaacaggggaggggaggagctagggaaagagaacctggagtttgtggcagggcttttgggattaagttcttcattcactaaggaaggaattgggaacactaagggtgggggcaggggagtttggggcaactggttggagggaaggtgaagttcaatgatgctcttgattttaatcccacatcatgtatcacttttttcttaaataaagaagcctgggacacagtaaaaaaaaaaaaaaaa

SEQ ID NO: 6 - DNA, 5084 bp, Homo sapiens
gatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttgccatactttgtctgaaaaattcctttgtgtttctattgacttcaatgatagtaagaaaagtggttgttagttatagatgtctaggtacttcaggggcacttcattgagagttttgtcaatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattc

SEQ ID NO: 7 - DNA, 23 bp, Artificial
  synthetic sequence
tatgcttcggtccacagagcata

SEQ ID NO: 8 - DNA, 25 bp, Artificial
  synthetic sequence
cacgtgacaagcgcaccgatacgtg

SEQ ID NO: 9 - DNA, 20 bp, Artificial
  primer
tccaggaacggaaaatcaag

SEQ ID NO: 10 - DNA, 20 bp, Artificial
  primer
tagagacctccgtcgctgtt

SEQ ID NO: 11 - DNA, 24 bp, Artificial
  primer
tgcagccaaagtgaagagggaaga

SEQ ID NO: 12 - DNA, 24 bp, Artificial
  primer
catagcgagcagcccaaagaagaa

SEQ ID NO: 13 - DNA, 24 bp, Artificial
  primer
tgcttgaatgtgctgatgacaggg

SEQ ID NO: 14 - DNA, 25 bp, Artificial
  primer
aaggcaagtcagcaggcatctcatc

SEQ ID NO: 15 - DNA, 22 bp, Artificial
  primer
gaacaatcccggtgtgtcagga

SEQ ID NO: 16 - DNA, 22 bp, Artificial
  primer
cacccagtggcagccttttaca

SEQ ID NO: 17 - DNA, 20 bp, Artificial
  primer
ccccacgacaaccgcaccat

SEQ ID NO: 18 - DNA, 20 bp, Artificial
  primer
cactccggcccacaaatctc

SEQ ID NO: 19 - DNA, 17 bp, Artificial
  primer
gcacccatggcagaagg

SEQ ID NO: 20 - DNA, 21 bp, Artificial
  primer
ctcgattggatggcagtagct

SEQ ID NO: 21 - DNA, 21 bp, Artificial
  primer
ggtttttaatcatcctcctcc

SEQ ID NO: 22 - DNA, 18 bp, Artificial
  primer
agcattgcccgacttctt

SEQ ID NO: 23 - DNA, 19 bp, Artificial
  primer
atgcaccgctacgacgtga

SEQ ID NO: 24 - DNA, 20 bp, Artificial
  primer
cttttgcacccctcccattt

SEQ ID NO: 25 - DNA, 25 bp, Artificial
  primer
acatcaaagctctgcagaaagaact

SEQ ID NO: 26 - DNA, 25 bp, Artificial
  primer
ctgaataccttcccaaatagaaccc

SEQ ID NO: 27 - DNA, 20 bp, Artificial
  primer
gttactgggcggagttcgta

SEQ ID NO: 28 - DNA, 20 bp, Artificial
  primer
tgaagtggcttggtgtcttg

SEQ ID NO: 29 - DNA, 18 bp, Artificial
  primer
acgaggcccagagcaaga

SEQ ID NO: 30 - DNA, 22 bp, Artificial
  primer
tcaggcagctcatagctcttct

(19) United States
(12) Patent Application Publication
(10) Pub. No.: US 2011/0158966 A1
(43) Pub. Date: Jun. 30, 2011

(54) STEM CELLS CHARACTERIZED BY EXPRESSION OF GERMLINE SPECIFIC GENES

(76) Inventor: Judith Seligman, Ra'anana (IL)

(21) Appl. No.: 13/029,975

(22) Filed: Feb. 17, 2011

Related U.S. Application Data
(63) Continuation-in-part of application No. 11/041,070, filed on Jan. 21, 2005, now abandoned.
Application No. 11/041,070 is a continuation of application No. PCT/IL03/00605, filed on Jul. 23, 2003.
Application No. PCT/IL03/00605 is a continuation-in-part of application No. 12/295,580, filed on Jul. 14, 2010.
(60) Provisional application No. 60/397,601, filed on Jul. 23, 2002.
(60) Provisional application No. 60/790,781, filed on Apr. 11, 2006.

(51) Int. Cl. A61K 35/14; C12N 5/0789; C12Q 1/68; A61P 1/16; A61P 15/00; A61P 9/00; A61P 19/08; A61P 25/00

(52) U.S. Cl. 424/93.7; 435/325; 435/366; 435/372; 435/6.1

(57) ABSTRACT

The present invention relates to adult stem cells derived from somatic sources characterized by the expression of the germline specific gene, DAZL. The somatic stem cells expressing the DAZL marker are further characterized by expression of additional markers and absence of expression of certain blood markers. In particular, the present invention discloses therapeutic and diagnostic uses, other than the germ cell potential use, of the DAZL multipotent stem cells isolated from somatic sources such as peripheral blood, bone marrow or umbilical cord blood.

### CROSS-REFERENCE TO RELATED APPLICATIONS

This application is a continuation-in-part of application Ser. No. 11/041,070 filed Jan. 21, 2005, which is a continuation of International Application no. PCT/IL2003/000605 filed Jul. 23, 2003, which claims the benefit of provisional application No. 60/397,601 filed Jul. 23, 2002. This application is also a continuation-in-part of application Ser. No. 12/295,580 filed Jul. 14, 2010, which is the 371 national stage filing of International Application no. PCT/IL2007/000467 filed Apr. 11, 2007, which claims the benefit of provisional application No. 60/790,781 filed Apr. 11, 2006. The entire content of each earlier filed application is expressly incorporated herein by reference thereto.

### FIELD OF THE INVENTION

The present invention relates to stem cells derived from somatic origins characterized by means of specific markers comprising gene products previously known as germline specific. The present invention specifically relates to isolation, characterization and uses of stem cells from peripheral blood, bone marrow or umbilical cord blood that express the germline specific gene, DAZL.

### BACKGROUND OF THE INVENTION

Embryonic stem (ES) cells are derived from the inner cell mass of a pre-implantation embryo and are pluripotent (Evans, M. J. & Kaufman, M. H. (1981) Nature 292, 154-156, Thomson et al. (1998) Science 282, 1145-1147), possessing the capability of developing into any organ or tissue type or, at least potentially, into a complete embryo. This has been shown by differentiating cells in vitro and by injecting human cells into immunocompromised (SCID) mice and analyzing resulting teratomas as disclosed in U.S. Pat. No. 6,200,806. Pluripotent embryonic stem cells have been shown to be able to differentiate into various cell types including cardiomyocytes, Hepatocytes, Neurons and germ cells (Geijsen et al., 2003, Clark et al., 2004 and Nayernia et al, 2006).

The fact that they are immortal and their ability to form all three embryonic layers make them especially suitable for therapeutic applications (Lerou, P. H. & Daley, G. Q. (2005) Blood Rev. 19, 321-331). Such use of ES cells, however, harbors major biological, ethical and legal problems, while adult stem cells isolated from blood and various tissues do not and thus may provide an alternative and equally efficacious source (Jensen, G. S. & Drapeau, C. (2002) Med. Hypotheses. 59, 422-428).

Adult Stem cells can also give rise to a succession of mature functional cells. For example, hematopoietic stem cells are multipotent and may give rise to any of the different types of terminally differentiated blood cells. Blood stem cells serve in the treatment of various diseases such as lymphomas and leukemias, as well as other neoplastic conditions where the stem cells are purified from tumor cells in the bone marrow or peripheral blood and re-infused into a patient after myelosuppressive or myeloablative chemotherapy.

Methods for separation and use of hematopoetic stem cells are known in the art. Characterizations and isolation of hematopoietic stem cells are reported in U.S. Pat. No. 5,061,620. The hematopoietic CD34 marker is the most common marker known to identify specifically blood stem cells, and CD34 antibodies are used to isolate stem cells from blood for transplantation purposes. However, CD34+ cells can differentiate only to blood cells and differ from embryonic stem cells that have the capability of developing into different body cells. Moreover, expansion of CD34+ cells is limited as compared to embryonic stem cells that are immortal. U.S. Pat. No. 5,677,136 discloses a method for obtaining human hematopoietic stem cells by enrichment for stem cells using an antibody, which is specific for the CD59 stem cell marker. The CD59 epitope is highly accessible on stem cells and less accessible or absent on mature cells. U.S. Pat. No. 6,127,135 provides an antibody specific for a unique cell marker (EM10) that is expressed on stem cells, and methods of determining hematopoietic stem cell content in a sample of hematopoietic cells. These disclosures are specific for hematopoietic cells and the markers used for selection are not absolutely absent on more mature cells.

Multiple tissue specific stem cell populations have also been found in various adult tissues, possessing limited differentiation potential. Neural stem cells were identified in the adult mammalian central nervous system (Ouredniket al. (1999) Clin. Genet. 56, 267-278), and adult stem cells have been identified from epithelial and adipose tissues (Zuk, et al. (2001) Tissue Eng. 7, 211-228). Finally, mesenchymal stem cells (MSCs) have been cultured from many parts of the body, including liver and pancreas, adipose tissues, muscle, brain and teeth (Jiang et al. (2002) Exp. Hematol. 30, 896-904, Hu et al. (2003) J. Lab. Clin. Med. 141, 342-349, Barry, F. P. & Murphy, J. M. (2004) Int. J. Biochem. Cell Biol. 36, 568-584).

The presence of multipotent progenitor cells in blood was suggested by in-vivo experiments following bone marrow and umbilical cord blood (UCB) transplantations into organs, such as brain, liver, pancreas and heart (Strauer et al. (2002) Circulation 106, 1913-1918, Ishikawa et al. (2003) Ann. N. Y. Acad. Sci. 996, 174-185, Zhaoet al. (2003) Brain Res. Protoc. 11, 38-45, Hess, et al. (2003) Nat. Biotechnol. 21, 763-770). However, such multipotent stem cells cannot be identified and isolated using the known markers.

Recent studies have demonstrated that certain somatic stem cells appear to have the ability to differentiate into cells of a completely different lineage. Monocyte- and mesodermal-derived cells that possess some multipotent characteristics have been identified, and stem cells possessing embryonic characteristics were also produced from UCB (McGuckinet al. (2005) Cell Prolif. 38, 245-255, Tondreau et al. (2005) Stem Cells 23, 1105-1112). Although these adult stem cells demonstrate some pluripotent potential, none of them possess proliferation and differentiation capabilities similar to those of ES cells. Furthermore, all the adult stem cell fractions studied so far were isolated and characterized by known blood markers (Pfendler, K. C. & Kawase, E. (2003) Obstet. Gynecol. Surv. 58, 197-208), all of which are membrane proteins of blood cells, and none of the cells was isolated by an embryonic marker. In some cases, fractionation and selection was made using varying culturing conditions, rendering these applications difficult to duplicate and standardize. U.S. Pat. No. 7,015,037 describes multipotent adult stem cells from bone marrow isolated by using CD90 and CD49C markers and by culturing conditions that allowed selecting cells possessing doubling rate of 36-48 hours.

There have been great efforts toward isolating pluripotent or multipotent stem cells, in earlier differentiation stages than hematopoietic stem cells, in substantially pure or pure form for diagnosis, replacement treatment and gene therapy purposes. Stem cells are important targets for gene therapy, where the inserted genes are intended to promote the health of the individual into whom the stem cells are transplanted. Stem cell-based therapies offer a promise for curing serious diseases for which no disease-modifying treatment options are available at present, such as Alzheimer's and Parkinson's diseases, paralysis due to spinal cord injury, heart failure, liver disease, and Type I diabetes.

### Embryonic Stem Cell and Germline Cell Gene Expression

Human ES cells express alkaline phosphatase, the stage-specific embryonic antigens SSEA-3 and SSEA-4, and surface proteoglycans that are recognized by the TRA-1-60 and TRA-1-81 antibodies. All these markers are not entirely specific to stem cells.

The molecular basis of pluripotency in stem cells is still unclear, with a few genes having been proposed to be involved in it, such as OCT-4, LIF/Stat3 and SOX-2 (Niwa, H. (2001) Cell Struct. Funct. 26, 137-148, Bortvin et al. (2003) Development 130, 1673-1680). Evidence suggesting that OCT-3/4 that is expressed specifically in pluripotent stem cells and regulates the fate of pluripotent stem cells was published (Niwa 2001 ibid, Niwa et al., 2000 (Nature Genetics 24, 372-376), and Zevnik et al., 1998 (Cell 95, 379-391)). It should be noted that OCT-3/4 also expresses in germ cells, however OCT-3/4 gene was not suggested to be directly involved in gametogenesis such as DAZL, but rather that it acts to maintain pluripotency.

Many other genes have been shown to be expressed in pluripotent ES cells (Sato et al. (2003) Dev. Biol. 260, 404-413, Bhattacharya et al. (2005) BMC Dev. Biol. 5, 22), including the germ stem cell-specific gene DAZL.

The DAZL gene, also known as DAZL1, DAZLA or DAZH, is an autosomal homolog of the DAZ (Deletion in Azoospermia) gene present on the Y chromosome (Saxena, R. et al. Nature Genet. 14, 292-299, 1996). These genes encode RNA binding proteins, first found to be expressed specifically in germ cells in the testis. Later studies have demonstrated that the DAZL gene expression is unique as it is expressed before meiosis in male and female gonads (Seligman and Page, Biochem. Biophys. Res. Com. 245, 878-82, 1998). Numerous genes are known to be expressed exclusively in male or female germ cells, mainly in meiotic or postmeiotic cells, but not in the earliest stages of gametogenesis. The expression of the human DAZL gene in both male and female germ cells so early during embryonic development is unusual. The TIAR gene, which is also an RNA-binding protein such as DAZL, was found to be expressed in primordial germ cells (Beck, A. R. P. et al. Proc. Natl. Acad. Sci. USA 95, 2331-2336, 1998). The DAZL gene is a germ cell specific gene and does not directly involved in the pluripotent mechanism: although disruption of DAZL in mice causes sterility, the animals were found to be otherwise normal. Moreover, the expression of DAZL and other germline specific genes declines as ES cells differentiate, and its expression is maintained only in the germline (Saxena et al. (1996) Nat. Genet. 14, 292-299, Geijsen et al. (2004) Nature 427, 148-154, Silverman, A. P. & Kool, E. T. (2005) Trends Biotechnol. 23, 225-230).

The identification of the DAZL marker in stem cells was based in part on the hypothesis that germ stem cells and ES cells have common expression patterns that are distinct from somatic cells. Indeed DAZL, Stellar, Nanog and Oct4 are expressed in germ stem cells, and not in differentiated somatic cells, and are expressed also in ES (Clark et al., Stem Cells 2004; 22:169-179). These genes may function with other genes in proliferation and maintenance of ES cells, in addition to their role in germ cell development. Genes and protein expressed in germ stem cells and in their progenitors, ES cells, and not expressed in differentiated somatic cells were used in WO 2004/009758, of the applicant of the present invention, to isolate stem cells from blood, tissue and organs.

U.S. Pat. Nos. 5,695,935; 5,871,920 and 6,020,476 disclose the nucleotide sequences of the DAZ gene family associated with azoospermia, while WO 02/10203 and US Patent Application Publication No. 2002165142 disclose four additional DAZ genes on the Y chromosome as well as isolated polypeptides encoded by these genes, antibodies to these polypeptides and methods for analyzing samples for the presence of the disclosed genes and their protein products.

PCT publication WO 2004/009758 to the applicant of the present invention, discloses that germline specific genes such as DAZL, that are also expressed in ES cells, can be used as markers to isolate stem cells from blood and other somatic tissues. Those stem cells possess broad differentiation potential, in addition to their germline potential. WO 2004/009758 discloses methods for identifying stem cell markers, such as DAZL, and uses thereof for identification, separation and characterization of stem cells from blood using these markers.

PCT publications WO 2006/001938, WO 2005/113752 and WO 2005/121321 disclose the use of bone marrow and peripheral blood derived germline stem cells and their progenitor cells to enhance or restore fertility in females. In these publications the population of the germline stem cells is defined by sets of germ cell markers, including DAZL.

There exists an unmet need for adult multipotent stem cells from somatic or peripheral sources, which can produce not only germ cells, but also other cell lineages, which are isolated from somatic cell populations. There exists also an unmet need for improved methods for identifying and isolating these cells which are highly valuable for cell therapy applications since they are easily available and free of legal, ethical and biological ramifications.

### SUMMARY OF THE INVENTION

The present invention provides for the first time characterized adult (including fetal) multipotent stem cells from somatic peripheral sources, such as peripheral blood, bone marrow, placenta or umbilical cord blood, that express the germline specific gene, DAZL. The somatic-derived multipotent stem cells according to the present invention are characterized by a set of markers that were originally found in ES cells and in germ stem cells but not in blood and other tissues.

It is now disclosed for the first time that the cell population isolated by use of DAZL gene marker comprises viable cells and provide differentiated cells such as hematopoietic colonies. The isolated cell population has a significantly greater differentiation potential compared to UCB mononuclear cells. It is also disclosed for the first time that cell population isolated by use of DAZL gene marker are multipotent, demonstrating broad differentiation potential in culture. Differentiation into various cell types, including hepatocytes, bone/cartilage, neurons, epithelial and heart muscle in addition to germ cells is disclosed. The multipotent stem cells according to the present invention may be thus used in methods of improving organ function, tissue reconstitution and tissue regeneration in mammals.

According to the present invention multipotent adult stem cells other than those derived from known sources of germline or ES cells are characterized by specific expression patterns of genes comprising the DAZL, optionally in association with SOX-2 and absence of expression of CD133, CD34, CD38, CD3 and CD14. These markers can be used for efficiently identification, separation and isolation of multipotent stem cells from somatic sources including blood, organs, tissue culture and cell suspensions.

The present invention provides somatic-derived multipotent stem cells characterized by DAZL expression and methods of identifying, characterizing, separating and using these DAZL expressing cells for diagnosis, therapy and tissue engineering. In particular, the present invention provides multipotent stem cells isolated from peripheral blood, bone marrow, umbilical cord blood, placenta and cells mobilized by relevant growth factors including but not limited to colony stimulating factor (CSF), stem cell factor (SCF) and granulocyte-colony stimulating factor (G-CSF) mobilized cells.

Somatic derived cells of the present invention are adult cells which do not comprise cells of the inner cell mass of embryonic blastocysts. Adult stem cells include fetal stem cells.

The present invention further provides cell population comprising multipotent stem cells derived from blood or tissues that express the specific germline marker DAZL and optionally express at least one additional gene selected from the group consisting of Stellar, SOX-2, c-Kit and Oct-4.

According to one embodiment the somatic-derived cell population is enriched in multipotent stem cells that express the specific germline marker DAZL.

The present invention further provides methods and reagents for use in prenatal diagnosis and tissue engineering methods.

The present invention further provides specific set of markers that can be used for identification, separation and characterization of the valuable multipotent stem cells from somatic tissues and organs, overcoming the ethical and logistical difficulties in the currently available methods for obtaining embryonic stem cells. The present invention further overcomes the limitations of known methods for isolation and characterization of multipotent stem cells by providing for the first time a specific set of markers that react with the stem cells present within somatic tissue or organ of an organism and is capable of separating the cells i.e. from bone marrow cells, colony stimulating factor mobilized cells, placenta cells and UCB cells.

The present invention now discloses isolated multipotent stem cells which are characterized by DAZL and SOX-2 and by absence of expression of the blood markers CD34, CD133, CD3, CD38 and CD14. These DAZL cells are demonstrated herein to be multipotent by differentiation into various types of cells, such as hematopoietic cells, neurons, bone/cartilage, endothelial, cardiomyocyte and hepatocyte, in addition to their ability to differentiate into germ cells.

According to another embodiment, the isolated stem cells are multipotent.

According to a preferred embodiment of the present invention, the blood cells and tissue samples are of mammalian origin, more preferably human origin.

According to an additional embodiment, the stem cells are isolated from somatic sources. According to this embodiment, said somatic source is selected from the group consisting of: umbilical cord blood, placenta, bone marrow derived blood hematopoietic cell and colony stimulating factor mobilized cells.

According to a specific embodiment, the stem cells are isolated from umbilical cord blood.

According to another specific embodiment, the stem cells are isolated from bone marrow. According to a further embodiment, the stem cells are isolated from placenta or fetal origin. According to yet another specific embodiment, the stem cells are isolated from fetal origin within a maternal cell population for non-invasive prenatal diagnosis.

According to a further embodiment, the stem cells are isolated from amniotic fluid obtained by amniocentesis.

According to one embodiment of the present invention the multipotent stem cell expresses DAZL and the ES marker SOX-2, do not express blood stem cell markers CD34, CD133 and do not express blood differentiation markers CD3, CD38 and CD14. According to a specific embodiment, the DAZL-expressing multipotent stem cell are isolated by negative selection of cells which do not express CD34, CD133, CD3, CD38 and CD14 followed by DAZL positive selection.

According to another embodiment of the present invention the multipotent stem cell expresses DAZL and at lease one additional marker selected from group consisting of: Stellar, SOX-2, c-Kit and Oct-4.

According to one aspect of the present invention the multipotent stem cells are isolated using germline specific gene markers which are selected based on their selective expression in primordial germ cells and/or germ stem cells and their absence in differentiated somatic cells. Thus, genes and proteins expressed in germ stem cells and in their progenitors, ES cells, are used according to the present invention as selective markers for isolation of multipotent stem cells from blood, tissue and organs. According to this aspect isolated stem cells expressing the germline specific gene marker DAZL and optionally co-expressing SOX-2 are disclosed as well as novel methods for use of multipotent stem cells in peripheral blood and other organs.

According to a specific embodiment, the expression of the germline specific gene marker is tested using a molecular probe which is used to label and select the viable cell suspension expressing the specific markers.

According to yet another specific embodiment, the molecular probe is a molecular beacon probe.

According to another aspect of the present invention a method for separating, sorting and isolating of stem cells, from somatic sources is provides which comprises the steps of:
i. separating a mononuclear cell fraction from colony stimulating factor mobilized cells, bone marrow or umbilical cord blood cells;
ii. introducing at least one molecular probe targeting at least one specific gene marker into living cells;
iii. optional removing cells which do not express at least one of the markers CD133, CD34, CD3, CD38 and CD14;
iv. sorting of cells by means of a sorting methodology; and
v. isolating the stem cells expressing the specific gene marker.

According to a specific embodiment the at least one specific gene marker of (ii) is selected from the group consisting of DAZL, c-kit, Stellar, Sox-2 and Oct-4. According to yet another specific embodiment a first selection is performed using a molecular probe targeting the DAZL protein or DAZL RNA, and at least one additional selection cycles is performed using at least one additional molecular probe targeting at least one marker selected from group consisting of: c-kit, Stellar, Sox-2 and Oct-4.

According to a specific embodiment the method include a specifically designed molecular probe, which will target the DAZL gene and optionally at least one other gene from the specific set of markers.

According to another embodiment the sorting methodology of (iv) comprises at least one density gradient that concentrates fetal cells.

According to yet another embodiment the molecular probe is labeled with a detectable tracer.

According to another embodiment the cell population from which stem cells are selected or sorted out, is selected from the group consisting of peripheral blood, umbilical cord blood, body fluids, tissue samples, tissue cultures, bone marrow hematopoietic cells, organ samples, organ cultures, cell lines and cell cultures.

According to yet another embodiment the expression of the germline specific gene marker is tested using a reagent selected from a polyclonal antibody, a monoclonal antibody, an antibody fragment, a polynucleotide probe, an oligonucleotide probe.

According to an additional embodiment the antibody is specific to at least one epitope of the DAZL protein. According to this embodiment the antibody is used for identification, selection or characterization of multipotent stem cells from mammalian fluids or tissues.

According to an additional embodiment the antibody comprises at least the antigen binding portion of an immunoglobulin specifically recognizing and binding a polypeptide having at least 70% homology to SEQ ID NO:2 or a peptide fragment retaining antigenic specificity of at least one epitope of DAZL.

According to yet another embodiment the antibody comprises at least the antigen binding portion of an immunoglobulin specifically recognizing and binding a polypeptide having at least 80% homology to SEQ ID NO:2 or a peptide fragment retaining antigenic specificity of at least one epitope of DAZL.

According to yet another embodiment the antibody comprises at least the antigen binding portion of an immunoglobulin specifically recognizing and binding a polypeptide having at least 90% homology to SEQ ID NO:2 or a peptide fragment retaining antigenic specificity of at least one epitope of DAZL.

According to a further embodiment the antibody comprises at least the antigen binding portion of an immunoglobulin specifically recognizing and binding a polypeptide having at least 70% homology to SEQ ID NO:3 or a peptide fragment retaining antigenic specificity of at least one epitope of DAZL.

According to yet another embodiment the antibody comprises at least the antigen binding portion of an immunoglobulin specifically recognizing and binding a polypeptide having at least 80% homology to SEQ ID NO:3 or a peptide fragment retaining antigenic specificity of at least one epitope of DAZL.

According to yet another embodiment the antibody comprises at least the antigen binding portion of an immunoglobulin specifically recognizing and binding a polypeptide having at least 90% homology to SEQ ID NO:3 or a peptide fragment retaining antigenic specificity of at least one epitope of DAZL.

Somatic-derived multipotent stem cells, characterized by expression of the germline specific gene DAZL, isolated according to the methods of present invention may be maintained and expanded in tissue culture in an undifferentiated state. According to various embodiments, these cells can be induced to differentiate into different cell types.

According to a further aspect of the present invention, therapeutic uses, other than the germ cell potential use, of somatic-derived multipotent stem cells, characterized by expression of the germline specific gene DAZL, are disclosed.

According to one embodiment a method of treating a genetic disorder comprising use of a multipotent somatic stem cell according to the invention, is disclosed. According to a specific embodiment the method of treating a genetic disorder comprises (a) modifying at least one gene of at least one somatic stem cell by a gene transfer to correct a genetic defect or provide genetic capability naturally lacking in the stem cell; and (b) administering to a patient suffering from a genetic disorder at least one modified cell of (a).

According to another embodiment a method of treating a tissue disorder or deficient comprising administering to a patient in need thereof a somatic-derived multipotent stem cell according to the invention and providing conditions for differentiation of said cells into cells characterizing said tissue, thereby treating the individual suffering from the tissue disorder or deficient requiring cell or tissue replacement, is disclosed.

According to another embodiment a method of producing or regenerating a human tissue or organ, comprising administering to a patient in need thereof a somatic-derived multipotent stem cell according to the invention, is disclosed. The method of treating an individual suffering from a disorder requiring cell or tissue replacement comprises introducing at least one multipotent stem cell isolated from a somatic source, wherein the cells is characterized by expression of the germline specific gene DAZL, into a tissue of the individual associated with the disorder, thereby treating the individual suffering from the disorder requiring cell or tissue replacement. According to a more specific embodiment method of treating an individual suffering from a disorder requiring cell or tissue replacement comprises (a) subjecting at least one somatic-derived multipotent stem cell characterized by expression of the germline specific gene DAZL, to culturing conditions suitable for inducing cell proliferation, thereby obtaining an expanded stem cell population; and (b) introducing said expanded stem cell population into a tissue of the individual associated with the disorder, thereby treating the individual suffering from the disorder requiring cell or tissue replacement. According to various embodiments the disorder or disease is selected from the group consisting of: hematopoietic disease or disorder, neuronal disease or disorder, endothelial disease or disorder, cartilage or bone disease or disorder, liver disease or disorder and heart disease or disorder. According to specific embodiments the disorder is a genetic disorder.

According to a further aspect of the present invention, a method of studying stem cell differentiation comprising use of a somatic-derived multipotent stem cell is disclosed. According to a specific embodiment somatic-derived stem cell expansion in culture is monitored. According to a further embodiment the method for monitoring stem cell expansion comprises the steps of i) labeling of culture cell with DAZL specific marker; ii) assessing the percentage of labeled and unlabeled cell; and iii) determining the ratio between differentiated and undifferentiated cell fraction wherein unlabeled fraction represents undifferentiated cell fraction and labeled fraction represents differentiated cell fraction.

According to one embodiment, the isolated stem cells are used for gene therapy. Cells are modified by appropriate gene transfer to correct genetic defects or provide genetic capabilities naturally lacking in the stem cells or their progeny.

According to another embodiment, the isolated multipotent stem cells are used for cell therapy. Multipotent stem cells according to the present invention may be administered to the patient in a manner that permits them to graft to the intended tissue site and reconstitute or regenerate the functionally deficient area.

According to other aspect of the invention, a method of restoring fertility is provided which comprises administering to a patient in need thereof multipotent stem cells derived from a somatic source wherein the stem cells are characterized by specific expression pattern of genes comprising the DAZL, optionally in association with SOX-2 and absence of expression of CD133, CD34, CD38, CD3 and CD14. According to some embodiments of this aspect the stem cells are differentiated into germ cells. According to other embodiments the multipotent stem cells are transplanted directly into testis or ovaries.

According to yet another embodiment the separated stem cells are used to study stem cell differentiation. Stem cells having the capacity to differentiate to various cell types are useful in tissue engineering and regeneration techniques.

According to a further aspect of the present invention diagnostic uses of somatic-derived multipotent stem cells, characterized by expression of the germline specific gene DAZL, are disclosed.

According to one embodiment a method of diagnosis of genetic disorder or chromosomal abnormality in a fetus comprising stem cells according to the invention, is disclosed. According to a specific embodiment the method of diagnosis comprises the steps of:
i. selecting at least one stem cell derived from the fetus using the DAZL specific marker;
ii. producing a display of the chromosomes of the embryo; and,
iii. analyzing the displayed chromosomes.

According to one embodiment the at least one stem cell is derived from amniotic fluid obtained by amniocentesis.

Another aspect of the present invention is directed to a kit for isolation, enrichment and detection of multipotent cells within a specimen, said kit comprising:
i. at least one reagent to detect DAZL protein or DAZL RNA;
ii. optionally reagents to detect any one of the gene products selected from the group consisting of: Stellar, Sox-2, c-kit and Oct-4;
iii. instructions for labeling, sorting and enrichment of the cells; and optionally
iv. means for performing stem cell labeling, sorting and enrichment.

According to a specific embodiment the kit may also include a specifically designed molecular probe, which will target the DAZL gene and optionally at least one other gene from the specific set of markers.

According to another embodiment the kit further comprises reagents for genetic analysis of fetal and maternal cells.

According to another embodiment the means for performing stem cell labeling, sorting and enrichment comprise at least one density gradient that concentrates fetal cells.

According to yet another embodiment the reagent of (i) is labeled with a detectable tracer.

Essentially all of the uses known or envisioned in the prior art for stem cells can be accomplished with the isolated cells and methods of the present invention. These uses include diagnostic, prophylactic and therapeutic techniques.

### BRIEF DESCRIPTION OF THE DRAWINGS

FIG. 1: Analysis of DAZL expression by reverse transcriptase-polymerase chain reaction (RT-PCR). RNA samples were isolated from bone marrow (lane 1), granulocyte colony stimulating factor (G-CSF) mobilized mononuclear cells (lane 2), cord blood mononuclear cells (lane 3) and adult peripheral blood mononuclear cells (lane 4). A 328 bp fragment of the human DAZL transcript was amplified. The gene encoding β-actin was used as a reference reaction.

FIG. 2: Fluorescent micrographs of DAZL expression on umbilical cord blood (UCB) mononuclear cells. Fixed UCB mononuclear cells were labeled with molecular beacons probes. Fluorescent and light illumination (a, b and a′, b′, respectively) of cells labeled with DAZL (a, a′) and with random molecular probes (b, b′). Fluorescent micrographs are of similar exposure time. Positive DAZL-expressing cells are indicated by arrows.

FIG. 3: Flow cytometry analysis of DAZL expression in mononuclear cells. Fluorescence-activated cell sorter (FACS) graphs of fixed cells labeled with molecular probe probes. A. UCB) mononuclear cells labeled with DAZL probe; B. UCB mononuclear cells labeled with random probe; C. Adult peripheral blood mononuclear cells labeled with a DAZL probe. The percent of positive cells is shown in the insert.

FIG. 4: Flow cytometry analysis of blood membrane markers on DAZL-isolated cells. Fixed UCB mononuclear cells labeled with a DAZL probe were incubated with (b-f) and without antibodies (a). Cells were incubated with CD38, CD14, CD3 and CD133 antibodies conjugated to Phycoerythrin (PE) (b, c, d, e, respectively) and analyzed for PE (antibodies) and 6FAM (DAZL) fluorescent signals. Cells exhibiting double staining of PE and FAM were expected on the upper right side of the graph

FIG. 5: Analysis of live cells, labeled with a DAZL probe by transfection. A. A representative fluorescence-activated cell sorter (FACS) illustration of labeled UCB mononuclear cells. a, UCB mononuclear cells labeled with a random probe (Random); b. UCB mononuclear cells labeled with a DAZL probe (DAZL); c. Adult peripheral blood mononuclear cells labeled with a DAZL probe. The percent of positive cells of the total number of cells is shown in the insert. B. Micrographs of isolated cells. Representative fluorescent (right) and light micrographs (left) of cells labeled with a DAZL probe and isolated by flow sorting. About 10⁵ positive cells were isolated in each experiment with a cell viability of >95%.

FIG. 6: Progenitor colony-forming test. Micrographs of representative hematopoietic colonies grown in methylcellulose that were seeded with the isolated DAZL-labeled cells. a. Burst-forming unit-erythroid (BFU-E) colony; b. Colony-forming unit-granulocyte macrophage (CFU-GM) colony; c. Colony-forming unit granulocyte, erythroid, macrophage, megakaryocyte (CFU-GEMM) colony; d. Fixed cells isolated from a CFU-GEMM colony stained with Giemsa. Erythroid (ery) and macrophage (mac) cells are indicated by arrows; e. and f. Light and fluorescence illumination, respectively, of fixed cells isolated from a CFU-GEMM colony labeled with a DAZL probe.

FIG. 7: Analysis of gene expression in isolated DAZL-labeled cells. Expression by RT-PCR using RNA extracted from DAZL-labeled cells isolated by cell sorting. Fragments of β-actin (566 bp), DAZL (488 bp), Stellar (174 bp), Oct-4 (133 bp) and Sox-2 (437 bp) were amplified. The marker peqGOLD 100 BP DNA-Ladder (peqlab) was loaded on the gel (Marker).

FIG. 8: Analysis of DAZL expression in different cell fraction. Expression by RT-PCR using RNA extracted from CD117+, CD117−, CDs+ and CDs− cells. Fragments of DAZL (488 bp) and β-actin (566 bp) were amplified.

FIG. 9: DAZL-expressing cells are multipotent, demonstrating broad differentiation potential. A. DAZL-expressing cells form colonies; micrographs of DAZL-expressing cell colonies grown in methycellulose. B. DAZL-expressing cells differentiate into different cell types; micrographs of DAZL-expressing cells grown in differentiation conditions. C. RT-PCR analysis of RNAs isolated from DAZL-expressing differentiated (on the right) and undifferentiated (on the left) cell culture. Fragments of various tissue specific osteogenic, myogenic, endothelial, hepatic and neurogenic were amplified.

FIG. 10: DAZL expressing cells (DE) are more efficient in restoring ovarian function as compared to Bone Marrow in ovarian failure following chemotherapy model.

FIG. 11: A representative histology demonstrating ovarian restoration following a treatment with DAZL expressing cells.

FIG. 12: A treatment with isolated DAZL expressing (DE) cells increases fertility rate of mice with ovarian failure by 3-fold and their progeny size by 2-fold.

FIG. 13: A treatment with isolated DAZL expressing (DE) cells increases progeny size by 2-fold.

### DETAILED DESCRIPTION OF THE INVENTION

### Terminology and Definitions

An “organism” is an individual form of life, including a body made up of organs, organelles, or other parts that work together to carry on the various processes of life or a living complex adaptive system of organs that influence each other in such a way that they function in some way as a stable whole. The term “organism” includes fetal life from Gastrulation which is followed by organogenesis, when individual organs develop within the newly formed germ layers.

“Stem cells” are undifferentiated cells, which can give rise to a succession of mature functional cells.

“Embryonic stem (ES) cells” are cells of the inner cell mass of the embryonic blastocysts that are pluripotent, thus possessing the capability of developing into any organ or tissue type or, at least potentially, into a complete embryo.

“Adult stem cells” are stem cells derived from tissues, organs or blood of an organism, excluding the inner cell mass of the embryo.

The term “somatic-derived multipotent stem cells” refers to stem cells derived from somatic sources, meaning all organs, fluids or cells of the organism, excluding the testes and ovaries where germ cells are produced. These somatic-derived multipotent stem cells can differentiate into various cell lineages, including germ cells, and can be used for various diagnostic and therapeutic purposes.

“A germ cell (or germline) specific marker” is used to describe a marker that reacts with gene products expressed specifically in germ cells in ovaries or testes.

The term “primordial germ cells” (PGCs) is used to describe undifferentiated embryonic germ cells isolated over a period of time post-fertilization from anlagen or from yolk sac, mesenteries, or gonadal ridges of embryos/fetus. Gonocytes of later testicular stages also can be useful sources of PGCs. PGCs are the source from which embryonic germ cells are derived and are pluripotent.

The term “germ cell specific gene expression” and “germ cell selective gene expression” are used interchangeably and refer to genes that are expressed in testis or ovary germ cells and are absent in other organs, in differentiated somatic cell types.

The term “germ cell-specific gene” means a gene that is expressed specifically in germ cells and that it is absent in somatic cells. The description “expressed specifically in germ cells” means that its expression is mainly in germ cells. It may also be expressed in somatic cells of the gonads (testis and ovaries), and a residual expression may also be found in somatic tissues. The term “germ cell-specific gene” is also indicative of a gene that is involved primarily in gametogenesis.

As used herein, the term “pluripotent stem cells” refers to cell that are: (i) capable of indefinite proliferation in vitro in an undifferentiated state; (ii) maintain a normal karyotype through prolonged culture; and (iii) maintain the potential to differentiate to derivatives of all three embryonic lineages (endoderm, mesoderm, and ectoderm) even after prolonged culture.

The term “multipotent cells” refers to stem cells, which can give rise to a number of cell types of more than one lineage.

The term “somatic cell” refers to cells that are mitotically competent, excluding the cells of the gonads (testes and ovaries).

The term “somatic source” refers to all tissues, fluids and organs of an organism excluding the gonads (testes and ovaries).

The term cell fraction” and “cell population” are use interchangeably, and refer to a specific sub-set of cells.

The term “homology”, as used herein, refers to a degree of sequence similarity in terms of shared amino acid or nucleotide sequences. There may be partial homology or complete homology (i.e., identity).

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands.

As used herein in the specification and in the claims section that follows, the phrase “complementary polynucleotide sequence” or complementary DNA (cDNA) includes sequences which result from reverse transcription of a messenger RNA template using a reverse transcriptase or any other RNA dependent DNA polymerase. Such sequences can be subsequently amplified in vivo or in vitro using a DNA dependent DNA polymerase.

A partially complementary sequence that at least partially inhibits an identical sequence from hybridizing to a target nucleic acid is referred to using the functional term “substantially homologous.” A substantially homologous sequence or hybridization probe will compete for and inhibit the binding of a completely homologous sequence to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence that lacks even a partial degree of complementarity (e.g., less than about 30% identity). In the absence of non-specific binding, the probe will not hybridize to the second non-complementary target sequence.

The terms “stringent conditions” or “stringency”, as used herein, refer to the conditions for hybridization as defined by the nucleic acid, salt, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. below the melting temperature (Tm) of the probe to about 20° C. to 25° C. below the melting temperature). One or more factors may be varied to generate conditions of either low or high stringency different from, but equivalent to, the above listed conditions.

“Nucleic acid sequence” as used herein refers to an oligonucleotide, nucleotide, or polynucleotide, and fragments thereof, and to DNA or RNA of genomic or synthetic origin which may be single- or double-stranded, and represent the sense or antisense strand. “Fragments” are those nucleic acid sequences which are greater than 60 nucleotides than in length, and most preferably includes fragments that are at least 100 nucleotides in length.

The term “oligonucleotide” refers to a nucleic acid sequence of at least about 6 nucleotides to about 60 nucleotides, preferably about 10 to 50 nucleotides, and more preferably about 20 to 30 nucleotides, which can be used in PCR amplification or a hybridization assay, or a microarray. As used herein, oligonucleotide is substantially equivalent to the terms “amplimers”, “primers”, “oligomers”, and “probes”, as commonly defined in the art.

The terms “specific binding” or “specifically binding”, as used herein, refers to that interaction for example between a protein or peptide and a binding agent such as an antibody. The interaction is dependent upon the presence of a particular structure (i.e., the antigenic determinant or epitope) of the protein recognized by the binding molecule. For example, if an antibody is specific for epitope “A”, the presence of a protein containing epitope A (or free, unlabeled A) will reduce the amount of labeled A bound to the antibody.

The term “antigenic determinant”, as used herein, refers to that fragment of a molecule (i.e., an epitope) that makes contact with a particular antibody. When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′)₂ fragments, Fv fragments and scFv.

Antibodies according to the present invention can be produced by any recombinant means known in the art. Such recombinant antibodies include, but are not limited to, fragments produced in bacteria and non-human antibodies in which the majority of the constant regions have been replaced by human antibody constant regions. In addition, such “humanized” antibodies can be obtained by host vertebrates genetically engineered to express the recombinant antibody.

The antibodies according to the present invention are obtained by methods known in the art for production of antibodies or functional portions thereof. Such methods include, but are not limited to, separating B cells with cell-surface antibodies of the desired specificity, cloning the DNA expressing the variable regions of the light and heavy chains and expressing the recombinant genes in a suitable host cell. Standard monoclonal antibody generation techniques can be used wherein the antibodies are obtained from immortalized antibody-producing hybridoma cells. These hybridomas can be produced by immunizing animals with stem cells, and fusing B lymphocytes from the immunized animals, preferably isolated from the immunized host spleen, with compatible immortalized cells, preferably a B cell myeloma.

Methods for the generation and selection of monoclonal antibodies are well known in the art, as summarized for example in reviews such as Tramontano and Schloeder, (Methods in Enzymology 178, 551-568, 1989). A recombinant or synthetic DAZL or a portion thereof of the present invention may be used to generate antibodies in vitro. More preferably, the recombinant or synthetic DAZL of the present invention is used to elicit antibodies in vivo. In general, a suitable host animal is immunized with the recombinant or synthetic DAZL of the present invention or a portion thereof including at least one continuous or discontinuous epitope. Advantageously, the animal host is a mouse of an inbred strain. Animals are typically immunized with DAZL or portion thereof in a physiologically acceptable vehicle, and a suitable adjuvant, which achieves an enhanced immune response to the immunogen. By way of example, the primary immunization conveniently may be accomplished with DAZL or a portion thereof and Freund's complete adjuvant, said mixture being prepared in the form of a water-in-oil emulsion. Typically the immunization may be administered to the animals intramuscularly, intradermally, subcutaneously, intraperitoneally, into the footpads, or by any appropriate route of administration. The immunization schedule of the immunogen may be adapted as required, but customarily involves several subsequent or secondary immunizations using a milder adjuvant such as Freund's incomplete adjuvant. Antibody titers and specificity of binding can be determined during the immunization schedule by any convenient method including by way of example radioimmunoassay, or enzyme linked immunosorbant assay, which is known as the ELISA assay. When suitable antibody titers are achieved, antibody producing lymphocytes from the immunized animals are obtained, and these are cultured, selected and closed, as is known in the art. Typically, lymphocytes may be obtained in large numbers from the spleens of immunized animals, but they may also be retrieved from the circulation, the lymph nodes or other lymphoid organs. Lymphocytes are then fused with any suitable myeloma cell line, to yield hybridomas, as is well known in the art. Alternatively, lymphocytes may also be stimulated to grow in culture; and may be immortalized by methods known in the art including the exposure of these lymphocytes to a virus; a chemical or a nucleic acid such as an oncogene, according to established protocols. After fusion, the hybridomas ate cultured under suitable culture conditions, for example in multiwell plates, and the culture supernatants are screened to identify cultures containing antibodies that recognize the hapten of choice. Hybridomas that secrete antibodies that recognize the recombinant or synthetic DAZL are cloned by limiting dilution and expanded, under appropriate culture conditions. Monoclonal antibodies are purified and characterized in terms of immunoglobulin type and binding affinity.

The antibodies can be conjugated to other compounds including, but not limited to, enzymes, magnetic beads, colloidal magnetic beads, haptens, fluorochromes, metal compounds, radioactive compounds or drugs. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m (⁹⁹Tc) ¹²⁵I and amino acids comprising any radionuclides, including, but not limited to, ¹⁴C, ³H and ³⁵S.

The term DAZL refers to “DAZ-like autosomal” or “Deleted in azoospermia-like 1” for designation of a polynucleotide or amino acid sequence is interchangeable with any of the terms DAZL1, DAZLA and DAZH. The human DAZL gene is the polynucleotide sequence of GenBank Accession Number U21663. The cDNA sequence of the human DAZL mRNA (Saxena et al. Nat. Genet. 14, 292-299, 1996), is presented in SEQ ID NO:1, wherein the coding sequence spans nucleotides 217-1098. The derived amino acid sequence of the human DAZL protein is presented in SEQ ID NO:2.

The term Stellar or Stella refers to homo sapiens germ and embryonic stem cell enriched protein STELLA (STELLAR), also known as developmental pluripotency associated-3 (DPPA3), is normally expressed in germ cells and in human embryonic stem cells. It is a germ cell developmental gene, expressed at high levels in primordial germ cells and oocytes, but it is almost absent from adult testis. Embryos deficient in Stella gene expression are compromised in preimplantation development and rarely reach the blastocyte stage. The gene Stellar is not involved specifically in gametogenesis, but rather is implicated in early embryogenesis (Bortvin et al. (2004) BMC Developmental biology 4, 1-5). The human Stellar gene is the polynucleotide sequence of GenBank Accession Number XR_{—}000555, GI:854181. The sequence of the human Stellar, is presented in SEQ ID NO:3.

Sox family transcription factors play essential roles in cell differentiation, development, and sex determination. Sox-2 was previously thought to be the sole Sox protein expressed in mouse embryonic stem (ES) cells. Sox-2 associates with Oct3/4 to maintain self-renewal of ES cells. Sox-2 is expressed specifically in human undifferentiated pluripotent ES cells and in germ cells, similar to OCT-4 (Maruyama et al. J Biol Chem. (2005) 280, 24371-24379; Western et al. (2005) Stem Cells. 23, 1436-1442).

The human Sox-2 gene is the polynucleotide sequence of GenBank Accession Number Z31560. The cDNA sequence of the human Sox-2 mRNA is presented in SEQ ID NO:4.

The transcriptional factor variously known as Oct-3, Oct-4 and Oct-3/4 was discovered in the early nineties by Okamato et al., (1990, Cell 461-472), Scholer et al., and Rosner et al., (1990 Nature 345: 686-692). The gene encoding for this transcription factor is now known as Pou5f1. The factor, denoted Oct-4 in the present application, plays an important role in development. The sequence of the human Oct-4 mRNA is presented in SEQ ID NO:5.

c-Kit Human-KIT (Proc. Natl. Acad. Sci. U.S.A. 89 (5), 1587-1591 (1992)) is a type 3 transmembrane receptor for MGF (mast cell growth factor, also known as stem cell factor). Mutations in KIT are associated with gastrointestinal stromal tumors, mast cell disease, acute myelogenous lukemia, and piebaldism. The sequence of the human c-Kit mRNA (Accession no.: NM_{—}000222.1, GI:4557694) is presented in SEQ ID NO:6.

Certain abbreviations are used herein to describe this invention and the manner of making and using it. For instance, CSF refers to colony stimulating factor, DAZL refers to DAZ-like autosomal and to Deleted in azoospermia-like 1, DNA refers to deoxyribonucleic acid, ES refers to embryonic stem, FACS refers to fluorescence activated cell sorter, FITC refers to fluorescein isothiocyanate, G-CSF refers to granulocyte-colony stimulating factor, MBP refers to molecular beacon probe, MPB refers to mobilized peripheral blood, PCR refers to polymerase chain reaction, RNA refers to ribonucleic acid, RT-PCR refers to reverse transcriptase PCR.

### Isolation and Assessment of DAZL Cell Multipotency

The present invention is directed to multipotent adult stem cells isolated by use of gene markers specific to multipotent stem cells, from somatic sources including peripheral blood, placenta, fetal cells and cord blood.

It is now disclosed that markers previously thought to be confined to the germ cell lineages, including germ cell precursors, are useful in the identification, characterization, selection or isolation of adult multipotent stem cells from somatic sources.

As shown in WO 2004/009758 of the applicant of the present innovation, DAZL is expressed in embryonic stem cells and teratocarcinoma cells, but is not expressed in the differentiated cell lines tested. DAZL thus provides a highly selective marker for multipotent stem cell identification and isolation from various adult and fetal tissues.

As exemplified in the present invention, DAZL cells are identified in bone marrow, mobilized peripheral blood, umbilical cord blood and mononuclear peripheral blood by RT-PCR and by microscopic and FACS analysis of labeled cells. The examples suggest that multipotent stem cells may be identified and separated from a plurality of tissues including bone marrow, both adult and fetal, mobilized peripheral blood, blood, umbilical cord blood, embryonic yolk sac, fetal liver, and spleen, both adult and fetal. Bone marrow cells may be obtained from any known source, including but not limited to, ilium (e.g. from the hip bone via the iliac crest), sternum, tibiae, femora, spine, or other bone cavities.

To assess cell multipotency, viable cells labeled with a DAZL polynucleotide probe were isolated from G-CSF mobilized mononuclear peripheral blood. G-CSF-mobilized mononuclear cells may be obtained from any suitable human donor or are commercially available (for example from BioWhittaker Inc. Walkersville, Md. USA). Mononuclear fractions can also be isolated from peripheral blood of patients treated with G-CSF using standard protocols.

According to one embodiment, in order to obtain maximum amounts of viable cells, labeling of cells may be performed with a DAZL polynucleotide probe following transfection according to well known protocols (for example Pederson T, Nucleic Acids Res. 29, 1013-1016, 2001). Following labeling, cells are subjected to cell sorting techniques to collect labeled cells. The labeled cells isolated by this method may be maintained and expanded in tissue culture in an undifferentiated state. According to various embodiments, these cells can be induced to differentiate into different cell types using state of the art methods.

It is now disclosed for the first time that the cell fraction isolated by DAZL gene marker is viable and that it creates hematopoietic colonies in methylcellulose. The isolated fraction has a significantly greater differentiation potential compared to UCB mononuclear cells: they formed about 3.5 times more hematopoietic colonies compared to UCB mononuclear cells (Table 2). Given the fact that the isolated cell fraction is not a purified fraction of DAZL-expressing cells, a higher number of hematopoietic colonies in purified populations can be expected. The results indicate that the efficiency can be improved in light of the fact that purified CD34+ formed about twice the number of the colonies than isolated cells. Since the DAZL-labeled cells are expected to be earlier progenitors compared to CD34+, it is possible that they need other conditions to efficiently differentiate into blood cells as is seen in ES cells. The surprising discovery of a high percentage of CFU-GEMM colonies that contained multi-potential progenitors of at least two cell lineages in DAZL-enriched plates supports this assumption.

It is now also disclosed for the first time that the cell fraction isolated by DAZL gene marker is viable and that it creates various colonies in methylcellulose medium, some of the colonies look like embryoid bodies that are typical to ES cells. Similar to ES cells, spontaneous differentiation is promoted if cells are grown without a feeder layer in Iscove's MDM medium. It is also shown that cells can be encouraged to differentiate into different cell lineages by cytokines. Analysis of differentiated DAZL cells by RT-PCR demonstrates the potential of the cells to differentiate into different cell types such as neurons, bone/cartilage, hepatocytes, endothelial and heart muscle. These results suggest that DAZL cells possess differentiation potential similar to ES cells and are multipoint.

### Separation Methods

Separation of the stem cells according to the present invention may be performed according to various physical properties, such as fluorescent properties or other optical properties, magnetic properties, density, electrical properties, etc. Cell types can be isolated by a variety of means including fluorescence activated cell sorting (FACS), protein-conjugated magnetic bead separation, morphologic criteria, specific gene expression patterns (using RT-PCR), or specific antibody staining.

The use of separation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342).

Cells may be selected based on light-scatter properties as well as their expression of various cell surface antigens. The purified stem cells have low side scatter and low to medium forward scatter profiles by FACS analysis. Cytospin preparations show the enriched stem cells to have a size between mature lymphoid cells and mature granulocytes.

Various techniques can be employed to separate the cells by initially removing cells of dedicated lineage. Monoclonal antibodies are particularly useful. The antibodies can be attached to a solid support to allow for crude separation. The separation techniques employed should maximize the retention of viability of the fraction to be collected.

The separation techniques employed should maximize the retention of viability of the fraction to be collected. Various techniques of different efficacy may be employed to obtain “relatively crude” separations. Such separations are where up to 30%, usually not more than about 5%, preferably not more than about 1%, of the total cells present are undesired cells that remain with the cell population to be retained. The particular technique employed will depend upon efficiency of separation, associated cytotoxicity, ease and speed of performance, and necessity for sophisticated equipment and/or technical skill.

Procedures for separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique.

Techniques providing accurate separation include fluorescence activated cell sorters, which can have varying degrees of sophistication, e.g., a plurality of color channels, low angle and obtuse light scattering detecting channels, impedance channels, etc.

Other techniques for positive selection may be employed, which permit accurate separation, such as affinity columns, and the like. The method should permit the removal to a residual amount of less than about 20%, preferably less than about 5%, of the non-target cell populations.

The antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the remaining cells.

Conveniently, after substantial enrichment of the cells lacking the DAZL marker, generally by at least about 50%, preferably at least about 70%, the cells may now be separated by a fluorescence activated cell sorter (FACS) or other methodology having high specificity. Multi-color analyses may be employed, with the FACS which is particularly convenient. The cells may be separated on the basis of the level of staining for the particular antigens.

While it is believed that the particular order of separation is not critical to this invention, the order indicated is preferred. Preferably, cells are initially separated by a coarse separation, followed by a fine separation, with positive selection of one or more markers associated with the stem cells and negative selection for markers associated with lineage committed cells.

### Molecular Beacon Probes

Molecular Beacon Probes (MBPs, U.S. Pat. Nos. 5,925,517; 6,103,476; 6,150,097 and 6,037,130) are single-stranded oligonucleotide hybridization probes that form a stem-and-loop structure. The loop contains a probe sequence that is complementary to a target sequence, and the stem is formed by the annealing of complementary arm sequences that are located on either side of the probe sequence. A fluorophore is covalently linked to the end of one arm and a quencher is covalently linked to the end of the other arm. Molecular probes do not fluoresce when they are free in solution. However, when they hybridize to a nucleic acid strand containing a target sequence they undergo a conformational change that enables them to fluoresce brightly.

In the absence of targets, the probe is dark, because the stem places the fluorophore so close to the nonfluorescent quencher that they transiently share electrons, eliminating the ability of the fluorophore to fluoresce. When the probe encounters a target molecule, it forms a probe-target hybrid that is longer and more stable than the stem hybrid. The rigidity and length of the probe-target hybrid precludes the simultaneous existence of the stem hybrid. Consequently, the molecular probe undergoes a spontaneous conformational reorganization that forces the stem hybrid to dissociate and the fluorophore and the quencher to move away from each other, restoring fluorescence.

Molecular Beacon probes are particularly specific. They easily discriminate target sequences that differ from one another by a single nucleotide substitution. The reason that molecular probes are so lightly specific is that they can exist in two different stable physical states. In one state, the molecular probes are hybridized to their targets, and energy is stored in the probe-target helix. In the second state, the molecular probes are free in solution, and energy is stored in their stem helix. Molecular probes are designed so that their probe sequence is just long enough for a perfectly complementary probe-target hybrid to be more stable than the stem hybrid. Consequently, the molecular probes spontaneously form fluorescent probe-target hybrids. However, if as little as a single nucleotide in the target is not complementary to the probe sequence of the molecular probe, the probe-target helix would be less stable. In this situation, the stem helix of the molecular probe is more stable than the mismatched probe-target helix, and the molecular probes remain unhybridized. Thus, molecular probes can be thought of as “molecular switches” that are on their targets and brightly fluorescent when the targets are perfectly complementary to the probe, but remain off the targets and dark if the targets contain a mutation.

The DAZL MBP sequence used to exemplify the present invention was 5′/6FAM/TATGCTTCGGTCCACAGAGCATA/BHQ1/3′ (SEQ ID NO:7), contains a specific 15 bases complementary sequence for the DAZL transcript sequence (nucleotides 955-972 of SEQ ID NO:1). Other DAZL MBP can be generated by targeting different sequences of the DAZL gene. It is also possible to use different variations of molecular probes such as:
1. MBP with different quencher and/or fluorophore
2. MBPs whose fluorophores form a fluorescent resonance energy transfer (FRET) pair
3. molecular probe aptamers
4. o-methyl RNA probe
5. quenched auto-ligation (QUAL)

### Use of DAZL Expressing Stem Cells for Diagnostic and Therapeutic Purposes

Cord blood offers multiple advantages over actual adult or ES cells and may provide the most accessible noninvasive resource of stem cells. It was disclosed that viable DAZL-positive cells could be easily isolated from UCB. It is now disclosed that the DAZL-positive cells expressed the embryonic pluripotent markers OCT-4, STELLAR, c-kit and SOX-2 and form blood colonies under appropriate tissue culture conditions. Furthermore, it is now also disclosed that the DAZL positive cells possess broad differentiation potential to various cell lineages. These surprising discoveries suggest that these isolated cells are stem cells with multipotent characteristics and they may be of considerable value for various therapeutic applications.

### Cell Therapy

A significant challenge to the use of stem cells for therapy is to control growth and differentiation into the particular type of tissue required for treatment of each patient.

### Organ and Tissue Therapy Applications Using Undifferentiated Cells

US 2002/197240 describes a method of inducing tissue and/or organ repair in vivo without eliciting an immune response. The method includes the transplantation of undifferentiated stem cells into a recipient suffering from tissue and/or organ damage. Undifferentiated cells of the present innovation can be transplanted following isolation to induce tissue and/or to repair organ of a recipient suffering from tissue and/or organ damage.

US 2004/247574 describes methods for improving engraftment efficiency in stem cell transplants by improving stem cell homing to bone marrow. Cell according to the present invention can be used for inducing organ function, tissue reconstitution or regeneration in a human patient in need thereof. The cells are administered in a manner that permits them to graft to the intended tissue site and reconstitute or regenerate the functionally deficient area.

### Organ and Tissue Therapy Applications Using Differentiated Cell Cultures

U.S. Pat. No. 6,087,168 is directed to transdifferentiating epidermal cells into viable neurons useful for both cell therapy and gene therapy. Skin cells are transfected with a neurogenic transcription factor, and cultured in a medium containing an antisense oligonucleotide corresponding to a negative regulator of neuronal differentiation.

International Patent Publication WO 97/32025 proposes a method for engrafting drug resistant hematopoietic stem cells. The cells in the graft are augmented by a drug resistance gene (such as methotrexate resistant dihydrofolate reductase), under control of a promoter functional in stem cells. The cells are administered into a mammal, which is then treated with the drug to increase engraftment of transgenic cells relative to nontransgenic cells.

International Patent Publication WO 99/19469 refers to a method for growing pluripotent embryonic stem cells from the pig. A selectable marker gene is inserted into the cells so as to be regulated by a control or promoter sequence in the ES cells, exemplified by the porcine OCT-4 promoter.

International Patent Publication WO 00/15764 refers to propagation and derivation of embryonic stem cells. The cells are cultured in the presence of a compound that selectively inhibits propagation or survival of cells other than ES cells by inhibiting a signaling pathway essential for the differentiated cells to propagate. Exemplary are compounds that inhibit SHP-2, MEK, or the ras/MAPK cascade.

Differentiated cells of the present invention can be used for tissue reconstitution or regeneration in a human patient in need thereof. The cells are administered in a manner that permits them to graft to the intended tissue site and reconstitute or regenerate the functionally deficient area.

Differentiated cells of present invention can also be used for transplant therapy. For example, neural stem cells can be transplanted directly into parenchymal or intrathecal sites of the central nervous system, according to the disease being treated (U.S. Pat. No. 5,968,829). The efficacy of neural cell transplants can be assessed in a rat model for acutely injured spinal cord as described by McDonald et al. (Nat. Med. 5, 1410, 1999).

### Forming New Blood Vessels in Damaged Tissue

US 2005/147597 provides methods of forming new blood vessels in diseased or damaged tissue in a subject, methods of increasing blood flow to diseased or damaged tissue in a subject, and methods of increasing angiogenesis in diseased tissue in a subject, which methods comprise: a) isolating autologous bone marrow-mononuclear cells from the subject; and b) transplanting locally into the diseased or damaged tissue an effective amount of the autologous bone-marrow mononuclear cells, thereby forming new blood vessels in the diseased or damaged tissue. Also provided are methods of treating tissue in disease or injury by local transplantation with an effective amount of the autologous bone marrow-mononuclear cells so as to induce vascularization in such diseased tissue.

Cells of the present invention can be used for tissue reconstitution or regeneration in a human patient in need thereof. The cells are transplanted locally in a manner that permits them to graft to the intended tissue site and reconstitute or regenerate new blood vessels in diseased or damaged tissue.

### Cell Therapy Applications for Neuronal Disorders

US 2006/211109 describes improved methods for efficiently producing neuroprogenitor cells and differentiated neural cells such as dopaminergic neurons and serotonergic neurons from pluripotent stem cells, for example human embryonic stem cells. The neuroprogenitor cells and terminally differentiated cells of the present invention can be generated in large quantities, and therefore may serve as an excellent source for cell replacement therapy in neurological disorders such as Parkinson's disease.

Certain neural differentiated cells of the present invention may be designed for treatment of acute or chronic damage to the nervous system. For example, excitotoxicity has been implicated in a variety of conditions including epilepsy, stroke, ischemia, Huntington's disease, Parkinson's disease and Alzheimer's disease. Certain differentiated cells of this invention may also be appropriate for treating dysmyelinating disorders, such as Pelizaeus-Merzbacher disease, multiple sclerosis, leukodystrophies, neuritis and neuropathies. Appropriate for these purposes are cell cultures enriched in oligodendrocytes or oligodendrocyte precursors to promote remyelination.

### Cell Therapy Applications for Bone/Cartilage Injuries

EP 1760144 describes a cartilage and bone repair composition comprising a group of human mesenchymal stem cells that are differentiated to the chondro-osteogenic lineage, by means of the amplification thereof. The composition can be employed using implants in the area to be repaired or it can be employed directly by injecting the cells in suspension either at the site of the injury or into the systemic circulation for the widespread distribution thereof

US 2007/048381 describes methods for promoting growth of bone, ligament, or cartilage in a mammal. The methods comprise administering to said mammal a composition comprising a pharmacologically effective amount of a zvegf3 protein in combination with a pharmaceutically acceptable delivery vehicle. Also disclosed are methods for promoting proliferation or differentiation of osteoblasts, osteoclasts, chondrocytes, or bone marrow stem cells.

Cells according to the present invention can be transplanted directly with osteogenic stimulators or transplanted following in-vitro differentiation to chondro-osteogenic lineage for tissue regeneration in a human patient in need thereof.

### Cell Therapy Applications for Liver Disorders

Hepatocytes and hepatocyte precursors prepared according to the present invention can be assessed in animal models for ability to repair liver damage. One such example is damage caused by intraperitoneal injection of D-galactosamine (Dabeva et al., Am. J. Pathol. 143, 1606, 1993). Efficacy of treatment can be determined by immunohistochemical staining for liver cell markers, microscopic determination of whether canalicular structures form in growing tissue, and the ability of the treatment to restore synthesis of liver-specific proteins. Liver cells can be used in therapy by direct administration, or as part of a bioassist device that provides temporary liver function while the subject's liver tissue regenerates itself following fulminant hepatic failure.

### Cell Therapy Applications for Heart Disorders

WO 2004/065589 and US 2003/031651 describe methods for preparing cell for cell transplantation and transplantation to mammal heart tissue in higher yield, so that it can treat a disorder by unstable heart function.

WO 2006/017567 describes methods of customizing the biological activity (e.g. rhythmic firing rate) of cardiomyocytes derived from pluripotent or multipotent stem cells, followed by transplantation to modify cardiac functions in vivo (e.g. to augment or attenuate the heart rate by modifying the cellular excitability of recipient cells).

US 2005/031600 describes methods and compositions for treating damaged or scarred myocardial tissue, by transplanting mesenchymal stem cells into the damaged or scarred tissue.

Cells of the present invention can be used to treat heart disorders in a human patient in need thereof. Successful treatment will improve heart function as determined by systolic, diastolic, and developed pressure. Cardiac injury can also be modeled using an embolization coil in the distal portion of the left anterior descending artery (Watanabe et al., Cell Transplant. 7, 239, 1998), and efficacy of treatment can be evaluated by histology and cardiac function. Cardiomyocyte preparations embodied in this invention can be used in therapy to regenerate cardiac muscle and treat insufficient cardiac function (U.S. Pat. No. 5,919,449 and WO 99/03973).

### Cell Therapy Applications to Treat Infertility

US 2005/015824 and WO 03/046129 describe compositions and methods for the reproducible derivation of germ cells (oocytes and spermatogonia) from stem cells. Also provide are methods of use of the same in reproductive and therapeutic cloning protocols.

Cells of the present invention can be differentiated into germ cells and or transplanted directly into testis and ovaries to treat infertility in human patients in need thereof.

### Gene Therapy

Gene therapy refers to the transfer and stable insertion of new genetic information into cells for the therapeutic treatment of diseases or disorders. The foreign gene is transferred into a cell that proliferates to spread the new gene throughout the cell population. Thus stem cells, or pluripotent progenitor cells, are usually the target of gene transfer, since they are proliferative cells that produce various progeny lineages which will potentially express the foreign gene.

Multipotent stem cells according to the present invention may be used in gene therapy for the treatment of a variety of diseases, particularly genetic diseases. Genetic diseases associated with hematopoietic cells may be treated by genetic modification of autologous or allogeneic stem cells to correct the genetic defect. For example, diseases including, but not limited to, β-thalassemia, sickle cell anemia, adenosine deaminase deficiency, recombinase deficiency, recombinase regulatory gene deficiency, etc. may be corrected by introduction of a wild-type gene into the selected DAZL cells, either by homologous or random recombination. Other indications of gene therapy are introduction of drug resistance genes to enable normal stem cells to have an advantage and be subject to selective pressure during chemotherapy. Diseases other than those associated with hematopoietic cells may also be treated by genetic modification, where the disease is related to the lack of a particular secreted product including, but not limited to, hormones, enzymes, interferons, growth factors, or the like. By employing an appropriate regulatory initiation region, inducible production of the deficient protein may be achieved, so that production of the protein will parallel natural production, even though production will be in a different cell type from the cell type that normally produces such protein. It is also possible to insert a ribozyme, antisense or other message to inhibit particular gene products or susceptibility to diseases, particularly hematolymphotropic diseases.

Alternatively, one may wish to remove a particular variable region of a T-cell receptor from the T-cell repertoire. By employing homologous recombination, or antisense or ribozyme sequence which prevents expression, the expression of the particular T-cell receptor may be inhibited. For hematotrophic pathogens, such as HIV, HTLV-I and II, etc. the stem cells could be genetically modified to introduce an antisense sequence or ribozyme which would prevents the proliferation of the pathogen in the stem cell or cells differentiated from the stem cells.

Optionally, the progenitor cells obtained using the method of the present invention can be manipulated to express desired gene products. Gene therapy can be used to either modify a cell to replace a gene product, to facilitate regeneration of tissue, to treat disease, or to improve survival of the cells following implantation into a patient (i.e. prevent rejection). In this embodiment, the progenitor cells are transfected prior to expansion and differentiation. Techniques for transfecting cells are known in the art.

A skilled artisan could envision a multitude of genes which would convey beneficial properties to the transfected cell or, more indirectly, to the recipient patient/animal. The added gene may ultimately remain in the recipient cell and all its progeny, or may only remain transiently, depending on the embodiment. For example, genes encoding angiogenic factors could be transfected into progenitor cells isolated from smooth muscle. Such genes would be useful for inducing collateral blood vessel formation as the smooth muscle tissue is regenerated. It some situations, it may be desirable to transfect the cell with more than one gene.

In some instances, it is desirable to have the gene product secreted. In such cases, the gene product preferably contains a secretory signal sequence that facilitates secretion of the protein. For example, if the desired gene product is an angiogenic protein, a skilled artisan could either select an angiogenic protein with a native signal sequence, e.g. VEGF, or can modify the gene product to contain such a sequence using routine genetic manipulation (Nabel J. G. et al., Thromb Haemost. 70, 202-203, 1993). The desired gene can be transfected into the cell using a variety of techniques. Preferably, the gene is transfected into the cell using an expression vector. Suitable expression vectors include plasmid vectors, viral vectors (such as replication defective retroviral vectors, herpes virus, adenovirus, adenovirus associated virus, and lentivirus), and non-viral vectors (such as liposomes or receptor ligands).

The desired gene is usually linked to its own promoter or to a foreign promoter which, in either case, mediates transcription of the gene product. Promoters are chosen based on their ability to drive expression in restricted or in general tissue types, or on the level of expression they promote, or how they respond to added chemicals, drugs or hormones. Other genetic regulatory sequences that alter expression of a gene may be co-transfected. In some embodiments, the host cell DNA may provide the promoter and/or additional regulatory sequences. Cells containing the gene may then be selected for by culturing the cells in the presence of the toxic compound. Methods of targeting genes in mammalian cells are well known to those of skill in the art (U.S. Pat. Nos. 5,830,698; 5,789,215; 5,721,367 and 5,612,205).

The methods of the present invention may be used to isolate and enrich stem cells or progenitors cells that are capable of homologous recombination and, therefore, subject to gene targeting technology. Most studies in gene therapy have focused on the use of hematopoietic stem cells. Recombinant retrovirus vectors have been widely used experimentally to transduce hematopoietic stem and progenitor cells. Genes that have been successfully expressed in mice after transfer by retrovirus vectors include human hypoxanthine phosphoribosyl transferase (Miller, A., et al. Science 255, 630, 1984). Bacterial genes have also been transferred into mammalian cells, in the form of bacterial drug resistance gene transfers in experimental models. The transformation of hematopoietic progenitor cells to drug resistance by eukaryotic virus vectors has been accomplished with recombinant retrovirus-based vector systems (Hock, R. A. and Miller, A. D. Nature 320, 275-277, 1986; Dick, J. E., et al. Cell 42, 71-79, 1985; Eglitis, M., et al., Science 230, 1395-1398, 1985). Recently, adeno-associated virus vectors have been used successfully to transduce mammalian cell lines to neomycin resistance (Tratschin, J. D. et al. Mol. Cell. Biol. 5, 3251, 1985). Other viral vector systems that have been investigated for use in gene transfer include papovaviruses and vaccinia viruses (see Cline, M. J. Pharmac. Ther. 29, 69-92, 1985).

Other methods of gene transfer include microinjection, electroporation, liposomes, chromosome transfer, and transfection techniques such as calcium-precipitation transfection technique to transfer a methotrexate-resistant dihydrofolate reductase (DHFR) or the herpes simplex virus thymidine kinase gene, and a human globin gene into murine hematopoietic stem cells. In vivo expression of the DHFR and thymidine kinase genes in stem cell progeny was demonstrated (Salser, W., et al. in Organization and Expression of Globin Genes, Alan R. Liss, Inc., New York, pp. 313-334, 1981).

Gene therapy has also been investigated in murine models with the goal of enzyme replacement therapy. Normal stem cells from a donor mouse have been used to reconstitute the hematopoietic cell system of mice lacking beta-glucuronidase (Yatziv, S. et al. J. Lab. Clin. Med. 90, 792-797, 1982). By this way, a native gene was being supplied and no recombinant stem cells (or gene transfer techniques) were needed.

### Cryopreservation

The freezing of cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroys the cell. These injurious effects can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, and inorganic salts.

In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After addition of DMSO, cells should be kept at 0° C. until freezing, since DMSO concentrations of about 1% are toxic at temperatures above 4° C.

A controlled slow cooling rate is critical. Different cryoprotective agents and different cell types have different optimal cooling rates (Lewis, J. P., et al. Transfusion 7, 17-32, 1967). The heat of fusion phase where water turns to ice should be minimal. The cooling procedure can be carried out by use of, e.g., a programmable freezing device or a methanol bath procedure. Programmable freezing apparatuses allow determination of optimal cooling rates and facilitate standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1 to 3° C./minute from 0° C. to −80° C. In a preferred embodiment, this cooling rate can be used for the neonatal cells of the invention. The container holding the cells must be stable at cryogenic temperatures and allow for rapid heat transfer for effective control of both freezing and thawing. Sealed plastic vials (e.g., Nunc, Wheaton cryules) or glass ampules can be used for multiple small amounts (1-2 ml), while larger volumes (100-200 ml) can be frozen in polyolefin bags (e.g., Delmed) held between metal plates for better heat transfer during cooling. (Bags of bone marrow cells have been successfully frozen by placing them in −80° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred aspect, DMSO-treated cells are precooled on ice and transferred to a tray containing chilled methanol which is placed, in turn, in a mechanical refrigerator (e.g., Harris or Revco) at −80° C. Thermocouple measurements of the methanol bath and the samples indicate the desired cooling rate of 1 to 3° C./minute. After at least two hours, the specimens have-reached a temperature of −8° C. and can be placed directly into liquid nitrogen (−196° C.) for permanent storage.

After thorough freezing, cells can be rapidly transferred to a long-term cryogenic storage vessel. In a preferred embodiment, samples can be cryogenically stored in liquid nitrogen (−196° C.) or its vapor (−165° C.). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

Considerations and procedures for the manipulation, cryopreservation, and long-term storage of hematopoietic stem cells, particularly from bone marrow or peripheral blood, are largely applicable to the neonatal and fetal stem cells of the invention (Gorin, N. C. Clinics In Haematology 15, 19-48, 1986)

Other methods of cryopreservation of viable cells, or modifications thereof, are available and envisioned for use (e.g., cold metal-mirror techniques; U.S. Pat. No. 4,199,022; U.S. Pat. No. 3,753,357; U.S. Pat. No. 4,559,298). U.S. Pat. No. 6,310,195 discloses a method for preservation of pluripotent progenitor cells, as well as totipotent progenitor cells based on a use of a specific protein. In a preferred case, the protein can preserve hematopoietic progenitor cells, but progenitor cells from other tissues can also be preserved, including nerve, muscle, skin, gut, bone, kidney, liver, pancreas, or thymus progenitor cells.

Frozen cells are preferably thawed quickly (e.g., in a water bath maintained at 37-41° C.) and chilled immediately upon thawing. In particular, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

### In Vitro Cultures of Hematopoietic Stem Cells and Progenitor Cells

An optional procedure (either before or after cryopreservation) is to expand the hematopoietic stem and progenitor cells in vitro. However, care should be taken to ensure that growth in vitro does not result in the production of differentiated progeny cells at the expense of multipotent stem and progenitor cells which are therapeutically necessary for hematopoietic reconstitution. Various protocols have been described for the growth in vitro of cord blood or bone marrow cells, and it is envisioned that such procedures, or modifications thereof, may be employed (Dexter, T. M. et al. J. Cell. Physiol. 91, 335, 1977; Witlock, C. A. and Witte, O. N. Proc. Natl. Acad. Sci. U.S.A. 79, 3608-3612, 1982).

WO 2006/085482 describes a technique for amplifying a hematopoietic stem cell ex vivo. By using the amplified hematopoietic stem cell or a stem cell of each of various tissues, a transplantation therapy and a gene therapy for a patient with a variety of intractable hematologic diseases or a variety of organ diseases can be conducted.

Various factors can also be tested for use in stimulation of proliferation in vitro, including but not limited to interleukin-3 (IL-3), granulocyte-macrophage (GM)-colony stimulating factor (CSF), IL-1 (hemopoietin-1), IL-4 (B cell growth factor), IL-6, alone or in combination.

The present invention further encompasses methods for obtaining compositions of cells which are highly enriched in stem cells. The method comprises incubating the compositions described above under conditions suitable for regeneration of stem cells. Compositions comprising the original stem cells and/or the regenerated stem cells are obtained thereby. Such a composition has utility in reconstituting human hematopoietic systems and in studying various parameters of hematopoietic cells as described above.

The invention also encompasses methods of use of the selected DAZL stem cell populations. The subject cell compositions may find use in any method known in the art. Since the cells are naive, they can be used to fully reconstitute an immunocompromised host such as an irradiated host or a host subject to chemotherapy; or as a source of cells for specific lineages, by providing for their maturation, proliferation and differentiation into one or more selected lineages by employing a variety of factors, including, but not limited to, erythropoietin, colony stimulating factors, e.g., GM-CSF, G-CSF, or M-CSF, interleukins, e.g., IL-1, -2, -3, -4, -5, -6, -7, -8, etc., or the like, or stromal cells associated with the stem cells becoming committed to a particular lineage, or with their proliferation, maturation and differentiation. The selected DAZL stem cells may also be used in the isolation and evaluation of factors associated with the differentiation and maturation of hematopoietic cells. Thus, the selected DAZL stem cells may be used in assays to determine the activity of media, such as conditioned media, evaluate fluids for cell growth activity, involvement with dedication of particular lineages, or the like.

### EXAMPLES

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

Experiments were performed to demonstrate that the stem cells according to the present invention can be isolated from blood, tissues and organs using specific gene markers, and that these isolated cells possess properties of multipotent stem cells.

### Methods

### Isolation of Mononuclear Cells

G-CSF mobilized, BM and UCB mononuclear cells were obtained from StemCell Technology Inc. and, in some cases, the cells were isolated from human UCB obtained from hospitals with the appropriate informed consent and ethics committee approval. For mononuclear isolation, cord blood was diluted 1:2 with Dulbecco's phosphate buffered solution (PBS) and mononuclear cells were separated using Ficoll-Histopaque (GE Healthcare Life Sciences) according to the manufacture's protocol. Cells were washed in RPMI medium containing 10% fetal bovine serum (FBS) (Sigma-Aldrich) and counted. Their viability was assessed by Trypan blue dye (Sigma-Aldrich), and experiments were performed when viability was higher than 95%.

### Labeling of Mononuclear Cells

MPs were synthesized by Genosys. The DAZL MP sequence was 5′/6FAM/TATGCTTCGGTCCACAGAGCATA/BHQ1/3′ (SEQ ID NO: 7), which is a specific 15-base target for the DAZL transcript sequences (nucleotides 955-972 of SEQ ID NO:1). The control “random” MP sequence was 5′/6FAM/CACGTGACAAGCGCACCGATACGTG/BHQ1 (SEQ ID NO: 8), whose specific 15-base target sequence does not match with any mammalian gene. MBPs were used to label cell suspension in fixed or unfixed conditions. In some experiments, the cells were fixed with 4% paraformaldehyde for 15 min on ice. They were then centrifuged at 1,200 rpm for 5 min and re-suspended in 1 ml RPMI medium containing 10% FBS and 0.2% Tween-20. The cells were incubated for 15 min at 37° C. and washed. Labeling with an MB probe was made in 1-ml cell suspension in RPMI medium with a final concentration of 0.2 μM per MB probe. The cells were incubated with the probe at room temperature (RT) for 30 min and washed with RPMI medium containing FBS before analyzed.

For labeling live cells, the MBPs were delivered into the living cells using TransIT oligo transfection reagent (Mirus, Madison Wis.), according to the manufacture's protocol. Briefly, serum-free RPMI medium was mixed with the transfection reagent and incubated for 20 min at RT. An MB probe at a final concentration of 0.2 μM was added to the transfection solution and incubated for an additional 20 min at RT. The transfection solution was then added to a 400-μl cell suspension (containing about 2×10⁷ cells) and incubated at 37° C. for 30 min. The cells were washed with RPMI medium containing 10% FBS before being analyzed by FACS.

In some experiments, the cells were mounted on slides using a Cytospin centrifuge. The slides were fixed with 50% methanol and 50% acetone solution for 5 min and dried. They were washed with RPMI medium containing 10% FBS and then reacted with an MB at a final concentration of 0.1 μM in the dark for 30 min at RT. After being washed, the labeled slides were examined by an Olympus epifluorescence microscope coupled to a CCD camera. A filter of 490 nm was used for 6FAM fluorescent detection.

### Flow Cytometry and Cell Sorting Analysis

DAZL-labeled mononuclear cells were reacted with various blood membrane antibodies conjugated to PE. The flow cytometry analysis was designed to detect double fluorescent signals on a cell in which one of the membrane antibodies was PE-labeled and the other was 6FAM-labeled. Antibodies against CD3 (Miltenyi Biotec), CD117, CD38, CD14 and CD113 (BioLegend) were used for flow cytometry analysis. Cells labeled with the antibodies without the DAZL probe and cells labeled with the DAZL probe without antibodies were analyzed as well.

Labeled cells were analyzed by FACScan (Becton Dickinson). Events ranging from 10,000-100,000 cells were acquired and data were analyzed using Cellquest software.

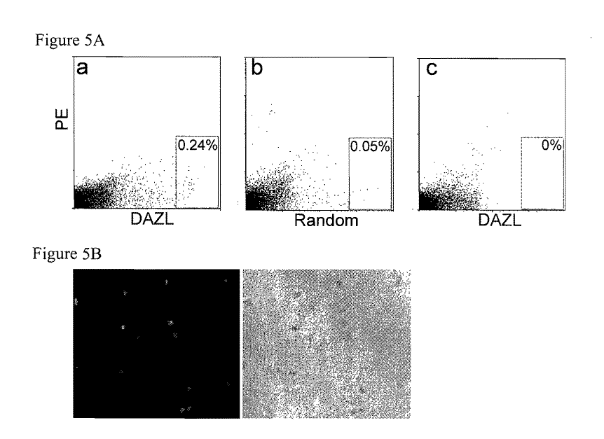

The cell sorting experiments were performed using a FACSVantage (Becton Dickinson) in sterile conditions. Live cells transfected with a DAZL probe were kept in RPMI medium containing 10% FBS during sorting. The sorting gate of positive cells exhibiting the highest fluorescent signals was 0.1-0.5% of the total analyzed cells (FIG. 5).

### Purification of Cell Fractions

Cells were purified from mononuclear fraction using Midimacs separation system (Miltenyi Biotec) according to the manufacturer's instructions. Briefly, CD34 cells were separated by direct labeling of cells with anti-human CD34-coated microbeads (CD34 MultiSort Kit). CD117 cells, CDs+ (positive to CD3, CD38 and CD14), CD− (negative to CD3, CD38 and CD14) were isolated using MACS Separator following indirect labeling. Cells were first labeled with the specific antibodies conjugated with PE, washed and labeled with anti-PE-coated microbeads. The labeled cells were passed twice through an LD Midimacs separation column in the MACS separator, and bound cells were gently flushed and collected for analysis.

### Colony-Forming Progenitor Assay

Human progenitor colony-forming assays were performed by planting cell populations at final concentrations of 1000 cells per ml into a 1-ml methylcellulose cocktail, Methocult GF+ H4435 (StemCell Technologies), containing 50 ng/ml recombinant human (rh) stem cell factor, 10 ng/ml rhGM-CSF, 10 ng/ml rhG-CSF, 10 ng/ml rhIL-3, 10 ng/ml rhIL-6, and 3 U/ml rh erythropoietin. The 35-mm methylcellulose plates were incubated at 37° C. with 5% CO2 in a humidified atmosphere. Differentiation into hematopoietic cell colonies was assessed, and colonies were inspected and scored two weeks later by an inverted microscope. Colonies were classified into CFU-GM, BFU-E and CFU-GEMM. Cells isolated from CFU-GEMM colonies were fixed on slides and stained with Giemsa (Sigma-Aldrich) to confirm the presence of erythroid cells and cells of at least two other recognizable lineages.

### Cell Culture

DAZL cells were grown in different conditions. In some experiments, cells were grown in Iscove's MDM (Bet Haemek, Israel) containing 10% Fetal Calf Serum (FCS), 1% penicillin/streptomycin (Gibco, Invitrogen) and 4 μl/ml Fibronectin (Bet Haemek, Israel). For expansion, cells were seeded in methylcellulose medium, MethoCult H4230 (StemCell Technologies Inc.), containing 1% methylcellulose in Iscove's MDM, 30% fetal bovine serum, 1% bovine serum albumin, 10⁻⁴ 2-mercaptoethanol and 2 mM L-glutamine. To promote differentiation, tissue cultures were supplemented with cytokines such as basic-FGF, VEGF, PDGF, NGF NOG and SCF.

### RT-PCR

Total RNA was extracted by Tri-reagent (Sigma) according to the manufacture's protocol. Synthesis of cDNA was performed using M-MLV reverse transcriptase (Promega) according to the manufacture's protocol. The reverse transcriptase products served as a template for independent PCR reactions. The following genes were amplified to analyze differentiation potential of DAZL-expressing cells to neurons—Nestin, to cartilage/bone—alkaline phosphatase (AP) and core binding factor alpha1 (Cbfa-1), to endothelial—vascular endothelial growth factor (VEGF), to heart muscle—myocyte enhancer factor 2C (MEF2C). The Sox-2, OCT-4, and Stellar and DAZL were also amplified. The β-actin gene was served as a control for RT-PCR reactions. The primer list and sequences are shown in Table 1.

TABLE 1
PCR primer list

| | | | SEQ ID |
|---|---|---|---|
| Gene | Primer (5′-3′) | Size | NO: |
| Nestin | F: TCCAGGAACGGAAAATCAAG | 210 bp | 9 |
| | R: TAGAGACCTCCGTCGCTGTT | | 10 |
| AP | F: TGCAGCCAAAGTGAAGAGGGAAGA | 216 bp | 11 |
| | R: CATAGCGAGCAGCCCAAAGAAGAA | | 12 |
| Albumin | F: TGCTTGAATGTGCTGATGACAGGG | 161 bp | 13 |
| | R: AAGGCAAGTCAGCAGGCATCTCATC | | 14 |
| MEF2C | F: GAACAATCCCGGTGTGTCAGGA | 452 bp | 15 |
| | R: CACCCAGTGGCAGCCTTTTACA | | 16 |
| Cbfa-1 | F: CCCCACGACAACCGCACCAT | 297 bp | 17 |
| | R: CACTCCGGCCCACAAATCTC | | 18 |
| VEGF | F: GCACCCATGGCAGAAGG | 90 bp | 19 |
| | R: CTCGATTGGATGGCAGTAGCT | | 20 |
| DAZL | F: GGTTTTTAATCATCCTCCTCC | 488 bp | 21 |
| | R: AGCATTGCCCGACTTCTT | | 22 |
| SOX-2 | F: ATGCACCGCTACGACGTGA | 437 bp | 23 |
| | R: CTTTTGCACCCCTCCCATTT | | 24 |
| OCT-4 | F: ACATCAAAGCTCTGCAGAAAGAACT | 133 bp | 25 |
| | R: CTGAATACCTTCCCAAATAGAACCC | | 26 |
| Stellar | F: GTTACTGGGCGGAGTTCGTA | 174 bp | 27 |
| | R: TGAAGTGGCTTGGTGTCTTG | | 28 |
| β-actin | F: ACGAGGCCCAGAGCAAGA | 566 bp | 29 |
| | R: TCAGGCAGCTCATAGCTCTTCT | | 30 |

Forward (F) and Reverse (R) primers of genes amplified by RT-PCR. The expected PCR product size of amplified cDNA is indicated.

### Example 1

### DAZL Express in Adult Peripheral Blood as Well as in Samples that are Rich in Hematopoietic Progenitor Stem Cells i.e., Bone Marrow, Granulocyte Colony Stimulating Factor Mobilized Cells and UCB Cells

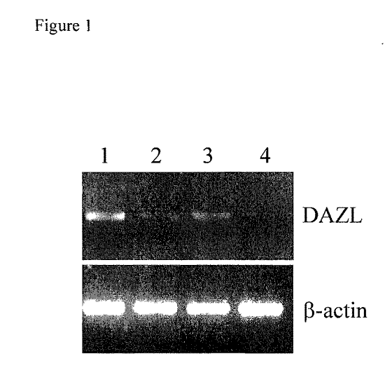

Expression of DAZL was identified in BM, in G-CSF mobilized cells and in UCB mononuclear cells by reverse transcriptase-polymerase chain reaction (RT-PCR) (FIG. 1). There was no significant expression of DAZL in adult peripheral mononuclear cells (FIG. 1 lane 4).

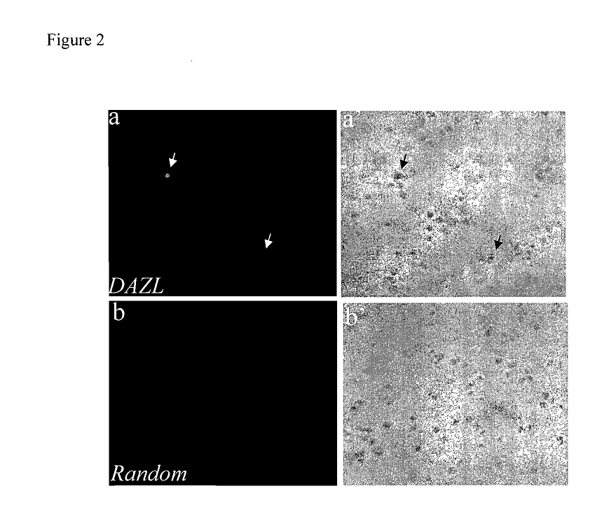

The expression of DAZL in mononuclear cells was localized by labeling the cells with a molecular beacon probe (MBP) that targets DAZL transcripts. The MBP probe contains a 6FAM fluorophore and a BHQ1 quencher and is designed to form a stem-loop hairpin structure that, in the absence of a target, quenches the fluorophore. Hybridization with a complementary target causes the hairpin to open, separating the fluorophore and the quencher, thereby restoring fluorescence. First UCB mononuclear cells were fixed on slides and reacted with a DAZL probe. As a control, a random MBP that targets a sequence that is not matched to any mammalian gene was used. The random probe was designed to contain a fluorophore and a quencher similar to the DAZL probe. Expression of DAZL was specifically detected in a few cells (FIGS. 2a &a′), and only some background expression was detected in controls labeled with the random MP (FIGS. 2b & b′). Similar results were obtained in BM and G-CSF mononuclear cells (not shown). Since UCB is a better resource of cells (i.e., more easily available and non-invasive) than G-CSF mobilized and BM, our current study focused on isolating and characterizing the cells from UCB.

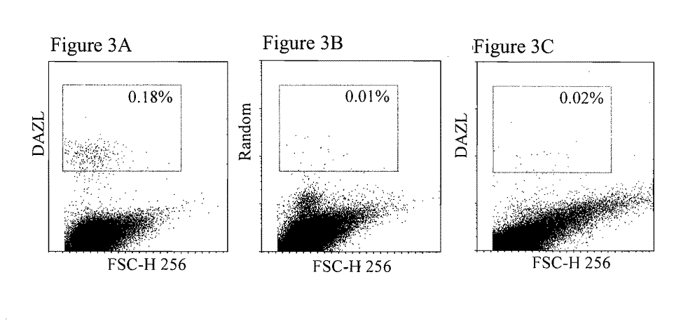

The percentage of DAZL-positive cells among the labeled cells was analyzed by flow cytometry. High fluorescence was demonstrated in a small fraction (0.18%) of the total UCB mononuclear cells labeled with the DAZL probe (FIG. 3A). A background fluorescence value of 0.01% of the total mononuclear cord blood was found in cells labeled with the random probe (FIG. 3B). Adult control peripheral blood mononuclear cells exhibited only the background fluorescence (FIG. 3C).

### Example 2

### Use of Molecular Beacon Probes to Isolate Cells

A molecular beacon probe (MBP) is an oligonucleotide that undergoes a conformational change upon hybridizing to a complementary target, resulting in a fluorescent signal. In its native state, the probe is a hairpin with the target sequence in the loop and a sequence that is non-complementary to the target in the stem. A fluorophore is attached to one end of the oligonucleotide, and a quencher is attached to the other terminus. MBPs are optimal for labeling live cells because they exhibit fluorescence upon binding to a target and can be rapidly degraded by cell nucleases with no long-term affect on the cells.

Different methods of molecular probes can be implemented to reduce reaction background, for example: using two MBPs whose fluorophores form a fluorescent resonance energy transfer (FRET) pair, 2′ o-methyl RNA probe, quenched auto-ligation (QUAL) (Silverman. & Kool. (2005) Trends Biotechnol. 23, 225-230, Tan et al. (2004) Curr. Opin. Chem. Biol. 8, 547-553. Fang et al. (2002) Cell Biochem. Biophys. 37, 71-81, Santangelo et al. (2004) Nucleic Acids Res. 32, e57)) etc.

MBPs are commonly in use for quantitative PCR (Tan et al. (2004) Curr. Opin. Chem. Biol. 8, 547-553) and were shown to be valuable for detecting real-time expression in living cells without damaging the cells or reducing their viability (Silverman. & Kool (2005) Trends Biotechnol. 23, 225-230).

MBPs can be designed to react with any gene of interest such as OCT-4 and germ cell specific genes. Following introduction of the MBP into the cells, labeled cells of interest can be isolated by FACS and implemented in various applications such as for cell culture, expression studies, therapy applications, diagnostics etc.

### Example 3

### Use of the MBP to Isolate DAZL Expressing Viable Cells

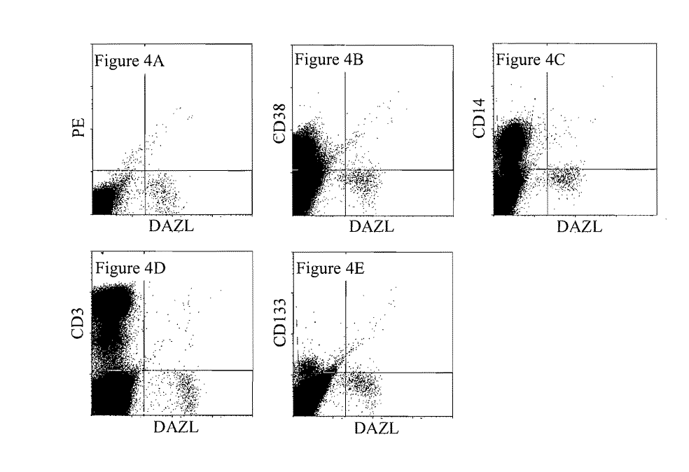

DAZL labeled cells were reacted with different blood membrane antibodies conjugated with Phycoerythrin (PE) in order to characterize the DAZL-positive ones. They were analyzed by flow cytometry to detect double fluorophore staining of 6FAM and PE. Minor double fluorescence of DAZL and antibodies was observed in each of the examined membrane markers (FIG. 4). The DAZL-positive cells thus appeared mostly negative to the blood markers CD38, CD14 and CD3, and mostly negative to the blood stem cell markers CD133 and CD34 (not shown). They were also negative to CD34, since no DAZL expression was detected in isolated CD34+ cells (not shown).

The MB probe was delivered into the cells by transfection in order to isolate viable cells expressing DAZL. Cell viability following transfection was over 95% as assessed by Trypan blue staining. Unlike the results following transfection in fixed cells (FIG. 3), the transfected cells exhibited different levels of fluorescence, with no distinct population of highly labeled cells (FIG. 5). The fluorescence in the DAZL-labeled cells (FIG. 5A), however, was significantly higher than the background fluorescence exhibited by the control random probe (FIG. 5B). As expected, adult peripheral blood mononuclear cells did not exhibit a significant signal above the control random probe (FIG. 5A-C). Cells that were sampled in the illustrated box of FIG. 5 were classified as being positive to DAZL and were isolated by cell sorting. All the positive cells that were sorted and isolated exhibited high levels of fluorescence, indicating that sorting was accurate, as can be seen by the fluorescence and light elimination of FIG. 5B.

### Example 4

### Use of Isolated DAZL-Positive Cells to Determine Whether the Act as a Stem Cells

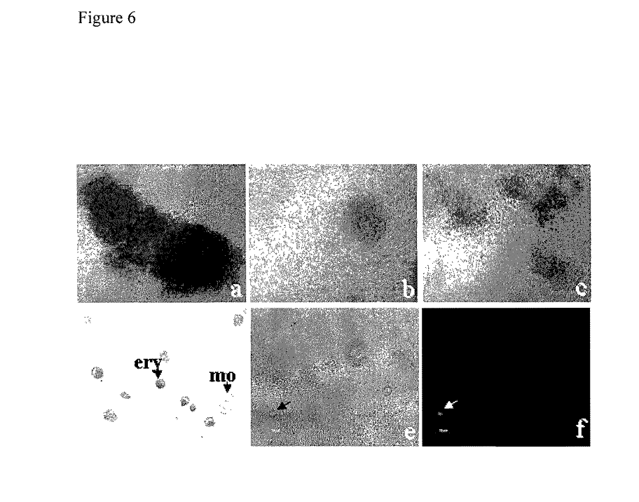

The isolated DAZL-positive cells were seeded in methylcellulose containing all the ingredients needed for hematopoietic cell growth in order to determine whether they act as stem cells. Mononuclear and CD34+ cells were used as a control reference and for assessing efficiency. All samples were seeded at concentrations of 1,000 cells per ml in methylcellulose. The total amount of hematopoietic colonies in plates seeded with DAZL-isolated cells was about 3.5-fold greater than the number of colonies counted in the control plates (21±4 compared to 6±1, respectively) (Table 2). The efficiency of CD34+ cells in forming hematopoietic colonies, however, was double that of the DAZL cell fraction, as assessed by the number of colonies in the CD34+ plates (53±8) and DAZL plates (21±4). The DAZL cell fraction formed various hematopoietic colonies, such as burst-forming unit-erythroid (BFU-E) (Table 2, FIG. 6a) and colony-forming unit-granulocyte macrophage (CFU-GM) (Table 2, FIG. 6b). About 3% of the hematopoietic colonies in the DAZL plates were progenitor colonies (i.e., comprised of colony-forming unit-granulocyte erythroid macrophages, CFU-GEMMs) (FIG. 6c) compared to 0% and 4% in the mononuclear and CD34+ plates, respectively (Table 2).

Cells from CFU-GEMM colonies were isolated and fixed on slides, and the presence of erythroid cells, monocytes and granulocytes was confirmed by Giemsa staining (FIG. 6d). We next examined whether DAZL expression persisted in cells of the hematopoietic colonies by reacting the slides with a DAZL probe. Microscopic examination of the slides revealed a small number of cells expressing DAZL in the colonies. A fluorescence DAZL-labeled cell among black cells negative to DAZL from a CFU-GEMM colony is presented in FIG. 6 (see light and fluorescent illumination, e & f, respectively). It appeared that not all progenitor DAZL cells differentiated, but that some also expanded in culture.

TABLE 2
The numbers of hematopoietic colonies on methylcellulose
plates seeded with control, DAZL and CD34 cells.

| | CONTROL | DAZL | CD34 |
|---|---|---|---|
| CFU-GM | 5 ± 0.5 (84%) | 18 ± 5 (85%) | 39 ± 2 (73%) |
| BFU-E | 1 ± 0.7 (16%) | 2 ± 1 (11%) | 12 ± 8 (23%) |
| CFU-GEMM | 0 ± 0 (0%) | 1 ± 1 (3%) | 2 ± 2 (4%) |
| Total | 6 ± 1 | 21 ± 4 | 53 ± 8 |

Mononuclear cells (Control), DAZL-positive isolated cell fraction (DAZL) and purified CD34+ cells (CD34) were seeded on methylcellulose plates at a concentration of 1,000 cells per ml. Colonies containing >200 cells were counted and classified into colony-forming unit-granulocyte macrophages (CFU-GMs), burst-forming unit-erythroids (BFU-Es) and colony-forming unit granulocyte, erythroid, macrophage, megakaryocytes (CFU-GEMMs). The results are presented as an average number of colonies per plate ± SD of three duplicated experiments.

### Example 5

### DAZL-Expressing Cells Possess Broad Differentiation Potential

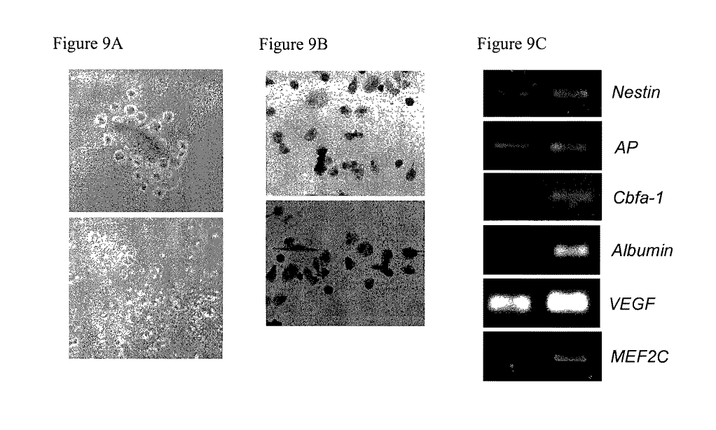

DAZL-expressing cells grown in methylcellulose without cytokines form colonies as can be seen in FIG. 9A). The colonies are different from blood colonies and some of them look like embryoid bodies that are typically to ES cells. In longer incubation conditions, differentiation of cells in colonies is seen. Differentiation is promoted if cells are grown in Iscove's MDM medium, cells grown in this condition demonstrate different morphologies that are typical to specific tissues, such as neurons and fat cells (see FIG. 9B). Cells can be encouraged to differentiate into different cell lineages by cytokines. Analysis of differentiated DAZL-expressing cells by RT-PCR demonstrates the potential of the cells to differentiate into different cell types. As can be seen in FIG. 9C, transcripts specific to neurons (Nestin), bone/cartilage (AFP and Cbfa-1), hepatocytes (Albumine), endothelial (VEGF) and heart muscle (MEF2C expression) were identified in differentiated DAZL-expressing cells. The results suggest that DAZL-expressing cells are multipotent, these cells have the potential to differentiate into a few cell lineages, in addition to their germline potential.

### Example 6

### Use of the DAZL Probe for Studying the Expression of Stem Cell-Specific Genes in DAZL Expressing Cells

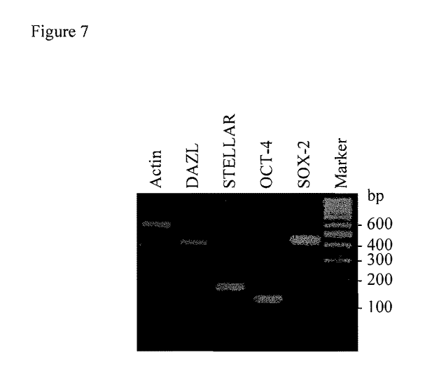

Expression of stem cell-specific genes in the cells isolated with a DAZL probe was analyzed to determine whether the DAZL-expressing cells also exhibited multipotent characteristics. RT-PCR analysis clearly demonstrated expression of DAZL, STELLAR, OCT-4 and SOX-2 genes in the isolated cell fraction (FIG. 7). Since STELLAR, OCT-4 and SOX-2 were expressed specifically in a pluripotent stage, this appeared to be evidence that the DAZL-isolated cells shared these characteristics of stem cells.

### Example 7

### Use of DAZL-Expressing Cells Isolated from Amniotic Fluid and Organs

DAZL-expressing cells are found in amniotic fluid, and other tissue organs. Cells from amniotic fluid are good source for fetal cells for women that undergo amniocentesis for prenatal diagnostic tests. The DAZL-expressing cells from the amniotic fluid may have better expansion and differentiation potential as compared to DAZL-expressing cells from blood. DAZL-expressing cells can also be isolated from organs, if organ sampling is performed to a patient as part of diagnostic or therapeutic procedure such as for Cardiac Catheterization.

### Example 8

### Use of Markers Associate with DAZL to Enrich DAZL-Expressing Cell Fraction

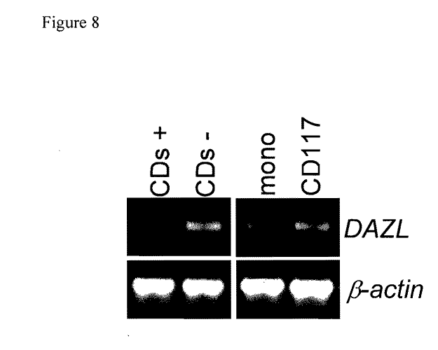

As was shown by FACS analysis, DAZL-expressing cells do not express membrane proteins specific to blood differentiated cells. It was shown that DAZL-expressing cells are negative to CD3, CD38 and CD14. To examine whether these markers are helpful for DAZL-expressing cell isolation, CDs− fraction (negative to CD3, CD38 and CD14) and CDs+ (positive to CD3, CD38 and CD14) were analyzed by RT-PCR and FACS. As can be seen in FIG. 8, DAZL is hardily detected in cells that are CDs+, most of DAZL expression is found in CDs− fraction that is negative to CD3, CD38 and CD14. The expression of DAZL in CDs− fraction is higher about 3-5 folds as compared to mononuclear cells (FIG. 8). Analysis by FACS support the RT-PCR results demonstrating that DAZL-expressing cells population was enriched about 5 folds as compared to mononuclear cells (1-2% as compared to 0.2-0.5% in CDs− and mononuclear, respectively). The addition of CD34 and CD133 to the negative selection (CDs−, negative to CD3, CD38, CD14, CD34 and CD133) slightly improves the enrichment results. Another option for DAZL-expressing cell enrichment is by using positive selection. However, most of the genes associated with DAZL are not expressed on the cell membrane. The CD117 (C-KIT) is a membrane protein that is associated with stem cells. The RT-PCR results of CD117 positive cell fraction suggest that DAZL-expressing cells are in CD117 positive fraction. The level of DAZL expression in CD117 cell fraction is similar to the level observed in CDs− fraction. Thus, the two options for enrichment of DAZL-expressing cell; the negative selection (CDs−) and the positive selection (CD117) may be used.

### Example 9

### Fertility Restoration in Mice

Chemotherapy induced ovarian failure model. Two groups of Balb/C female mice, 6 weeks old were injected intra peritoneum (IP) for chemotherapy with Cyclophosphamine/Busulfan (140/14 mg/kg, respectively). Group A did not receive a second therapy injection. Group B was administrated with a second chemotherapy injection 3 weeks following the first one. Ovarian function was assessed by counting the total oocytes numbers in serial cross sections of the ovaries that were fixed in Formalin 4 weeks following the last injection. As result, the total number of developing oocytes was decreased dramatically following chemotherapy as compared to controls (injected with saline solution). There were almost no oocytes with germinal vesicles (GV) in Group B. This indicates that chemotherapy protocol B induced a strict ovarian failure.

Bone Marrow cells were injected to the tail vain of chemotherapy induced ovarian failure mice (Group B) 4 weeks after the last chemotherapy injection. Total cell numbers of 2×107 or 2×106 in 100 ml PBS per animal were injected. Only 1 out of 13 treated animals demonstrated some effect of restoration reflected by the increase in total oocytes numbers indicating that restoration of ovarian function by Bone Marrow mononuclear cells is not efficient.

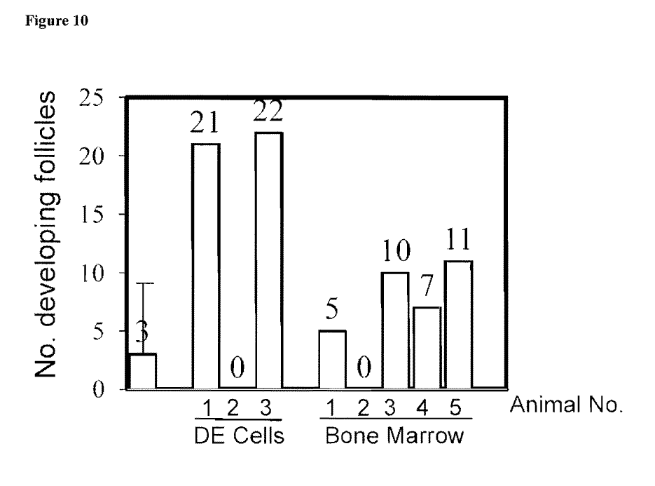

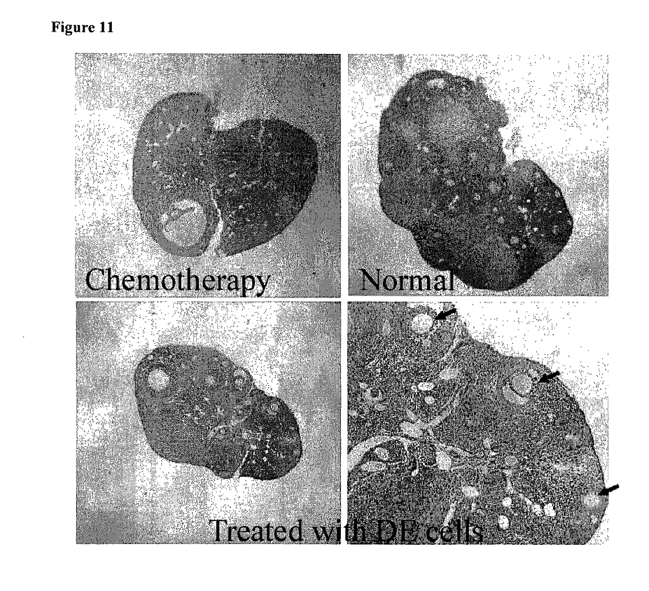

Isolated DAZL expressing cells (total 2×10⁶ cells) were injected in 100 ml PBS to the tail vain of chemotherapy induced ovarian failure mice (Group B) 4 weeks after the last chemotherapy injection. As can be seen in FIG. 10, two out of three animals treated with the isolated DAZL expressing cells demonstrated ovarian restoration as reflected by the increase in total oocytes numbers. All five animals treated at the same experiment with Bone Marrow cells did not demonstrate ovarian restoration. A representative histology demonstrating ovarian restoration following a treatment with DAZL expressing cells is shown in FIG. 11.

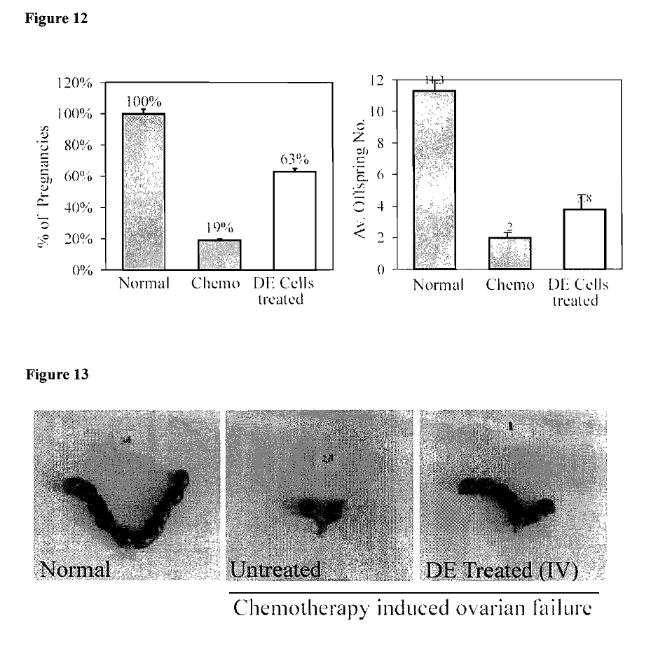

To examine whether ovarian histology results reflect fertility rate, females were bred with fertile males for a period of one month. Fertility rate and progeny size 12.5 dpc (days post coitum) were recorded. During one month of breeding, 100% of normal females were pregnant, while only 19% of chemotherapy treated mice were pregnant. A 3-fold increase in the pregnancy rate (63% pregnancies, FIG. 12) was observed in chemotherapy treated females following DAZL expressing (DE) cell transplantation. Progeny size of DE cell transplanted mice was double as compared to control chemotherapy treated mice (FIG. 13).

The results demonstrate that transplantation of DE cells can rescue ovarian failure in mice. Thus, this suggests a clinical potential of DE cell treatment for overcoming ovarian failure.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

What is claimed is:

1. An isolated stem cell derived from a somatic source, characterized by expression of the germline specific gene DAZL and at least one gene selected from the group consisting of: SOX-2, OCT-4, c-Kit and Stellar.

2. The isolated stem cell of claim 1 expressing the genes SOX-2, OCT-4, c-Kit and Stellar.

3. The isolated stem cell of claim 1 which does not express at least one of the blood markers selected from the group consisting of: CD3, CD38, CD14, CD34 and CD133.

4. The isolated stem cell of claim 1 wherein the cell is multipotent.

5. The isolated cell of claim 1, wherein the cell is a human cell.

6. The isolated cell of claim 5, wherein the cell is isolated from a cell population selected from the group consisting of: peripheral blood, umbilical cord blood, placenta, bone marrow, stem cell factor mobilized cells, and colony stimulating factor mobilized cells.

7. The isolated cell of claim 5, wherein the cell is isolated from amniotic fluid.

8. The isolated cell of claim 5, wherein the cell is of fetal origin within a maternal cell population.

9. A method of diagnosis of a genetic disorder or chromosomal abnormality in a fetus which comprises:
i. selecting at least one stem cell according to claim 1, from a fetus using the DAZL specific marker;
ii. producing a display of the chromosomes of the embryo; and
iii. analyzing the displayed chromosomes.

10. The method of claim 9 wherein the at least one stem cell is derived from amniotic fluid.

11. A method of treating a disorder or a disease comprising administering to a patient in need thereof a stem cell according to claim 1 and providing conditions for differentiation of said cells into cells characterizing the tissue, thereby treating the individual suffering from the tissue disorder or disease.

12. The method of claim 11 wherein the disorder or disease is selected from the group consisting of: hematopoietic disease or disorder, neuronal disease or disorder, endothelial disease or disorder, cartilage or bone disease or disorder, liver disease or disorder, fertility disease or disorder and heart disease or disorder.

13. The method of claim 11 which further comprises subjecting the somatic-derived stem cell to culturing conditions suitable for inducing cell proliferation, thereby obtaining an expanded stem cell population; and introducing said expanded stem cell population into a tissue of the individual associated with the disorder, thereby treating the individual suffering from the disorder requiring cell or tissue replacement.

14. The method of claim 11 which comprises direct administration of the stem cell into testis or ovaries thereby restoring fertility.

15. A method of isolating stem cells from a somatic source within an organism which comprises:
separating a mononuclear cell fraction from a cell population;
introducing a molecular probe targeting the specific marker DAZL and optionally at least one additional marker selecting from the group consisting of Sox-2, Stellar, Oct-4 and c-kit into the mononuclear cell fraction thus obtained;
sorting the cells by means of sorting methodology; and
isolating the stem cells expressing the specific marker DAZL and optionally the at least one of the genes selected from group consisting of: Sox-2, Stellar, Oct-4 and c-kit.

16. The method of claim 15 which further comprises removing cells expressing at least one of the blood markers selected from the group consisting of CD3, CD38, CD14, CD34 and CD133.

17. The method of claim 15, wherein the molecular probe is a molecular beacon probe.

18. The method of claim 15, wherein the cell population is selected from the group consisting of: peripheral blood, umbilical cord blood, bone marrow, stem cell factor mobilized cells, colony stimulating factor mobilized cells, a body fluid, a tissue sample, a tissue culture, an organ sample, an organ culture, a cell line and a cell culture.

19. A kit for isolation, enrichment and detection of somatic stem cells within a specimen, said kit comprising:
at least one reagent to detect DAZL protein or DAZL RNA;
optionally reagents to detect at least one of the genes or gene products selected from the group consisting of: Sox-2, Stellar, C-kit, and Oct-4;
instructions for labeling, sorting and enrichment of the cells; and optionally means for performing stem cell labeling, sorting and enrichment.

20. The kit of claim 19 further comprising reagents and means for negative selection of cells which express at least one of the blood markers: CD3, CD38, CD14, CD34 and CD133.

21. The kit of claim 19 further comprising reagents for genetic analysis of fetal and maternal cells.
